(19) United States
(12) Patent Application Publication
(10) Pub. No.: US 2010/0030340 A1
(43) Pub. Date: Feb. 4, 2010

(54) Plasticized Grafts and Methods of Making and Using Same

(76) Inventors: Lloyd Wolfinbarger, JR., Norfolk, VA (US); Katrina Crouch Ruth, Southaven, MS (US); Billy G. Anderson, Virginia Beach, VA (US); Darlea Anderson Mastin, (US)

Correspondence Address: MORGAN LEWIS & BOCKIUS LLP, 1111 PENNSYLVANIA AVENUE NW, WASHINGTON, DC, 20004, US

(21) Appl. No.: 12/404,855

(22) Filed: Mar. 16, 2009

Related U.S. Application Data
(63) Continuation-in-part of application No. 10/445,056, filed on May 27, 2003, now abandoned.
Application No. 10/445,056 is a continuation-in-part of application No. 09/874,862, filed on Jun. 5, 2001, now Pat. No. 6,569,200.
Application No. 09/874,862 is a division of application No. 09/107,459, filed on Jun. 30, 1998, now Pat. No. 6,293,970.
Application No. 09/107,459 is a continuation-in-part of application No. 11/826,522, filed on Jul. 16, 2007.
Application No. 11/826,522 is a continuation-in-part of application No. 10/694,190, filed on Oct. 28, 2003.
Application No. 10/694,190 is a continuation-in-part of application No. 09/660,422, filed on Sep. 12, 2000, now Pat. No. 6,743,574.
Application No. 11/826,522 is a continuation-in-part of application No. 10/624,534, filed on Jul. 23, 2003, now Pat. No. 7,338,757.
Application No. 10/624,534 is a continuation-in-part of application No. 09/528,371, filed on Mar. 17, 2000, now Pat. No. 6,734,018.
Application No. 09/528,371 is a continuation-in-part of application No. 09/327,240, filed on Jun. 7, 1999, now abandoned.

(51) Int. Cl. A61F 2/00

(52) U.S. Cl. 623/23.72

(57) ABSTRACT

The invention provides a plasticized tissue or organ that does not require special conditions of storage, for example refrigeration or freezing, exhibits materials properties that approximate those properties present in natural tissue, is not brittle, does not necessitate rehydration prior to clinical implantation and is not a potential source for disease transmission. Replacement of the chemical plasticizers by water prior to implantation is not required and thus, the plasticized bone or soft tissue product can be placed directly into an implant site without significant preparation in the operating room.

This application is a continuation-in-part application of U.S. patent application Ser. No. 10/445,056, filed May 27, 2003, which is a continuation-in-part application of U.S. patent application Ser. No. 09/874,862, filed on Jun. 5, 2001, now U.S. Pat. No. 6,569,200, which is a divisional application of U.S. patent application Ser. No. 09/107,459, filed on Jun. 30, 1998, now U.S. Pat. No. 6,293,970. This application is also a continuation-in-part application of U.S. patent application Ser. No. 11/826,522, filed Jul. 16, 2007, which is a continuation-in-part of U.S. patent application Ser. No. 10/694,190, filed Oct. 28, 2003, which is a continuation-in-part of U.S. patent application Ser. No. 09/660,422, filed Sep. 12, 2000, now U.S. Pat. No. 6,743,574. U.S. patent application Ser. No. 11/826,522, filed Jul. 16, 2007, is also a continuation-in-part of U.S. patent application Ser. No. 10/624,534, filed Jul. 23, 2003, now U.S. Pat. No. 7,338,757, which is a continuation-in-part of U.S. patent application Ser. No. 09/528,371, filed Mar. 17, 2000, now U.S. Pat. No. 6,734,018, which is a continuation-in-part of Ser. No. 09/327,240, filed Jun. 7, 1999, now abandoned. The above referenced applications are herein incorporated by reference in their entireties.

### FIELD OF THE INVENTION

The present invention provides a plasticized dehydrated tissue or organ graft that does not require special conditions of storage for example refrigeration or freezing, exhibits materials properties that approximate those properties present in normal hydrated tissue, or organ is not brittle and does not necessitate rehydration prior to clinical implantation. The graft may be a hard tissue, such as bone, a soft tissue, or an organ. Replacement of the chemical plasticizers by water prior to implantation is not required and thus, the plasticized graft can be placed directly into an implant site without significant preparation in the operating room. The present plasticized raft does not need rehydration, possesses adequate materials properties, and is not a potential source for disease transmission.

### BACKGROUND OF THE INVENTION

Bone tissue is a homogeneous material including osteoid and minerals. The osteoid is a viscous gel-like material including primarily type I collagen (approximately 90%), proteoglycans, and various sulfated and non-sulfated mucopolysaccharides. The mineral component consist primarily of a crystalline form of calcium phosphate, hydroxyapatite, with amounts of calcium carbonate, tricalcium phosphate, and smaller amounts of other forms of mineral salts. This bone tissue is laid down around cells called osteocytes and these cells are found in small interconnected channels (lacunnae) which are interconnected through a series of channels including the Haversian canal system. At the level of the microscope, it is possible to observe that bone tissue is organized into osteons of compact bone made of concentric, perivascular layers of highly coaligned mineralized collagen fiber bundles. The predominant orientation within a single layer varies with respect to the vascular axis and various combinations of orientation in successive lamellae and results in variable overall collagen orientation within each osteon. Differences in overall collagen orientation are directly reflected in differing mechanical behavior of single osteons. Transversely oriented collagen results in better resistance to compressive loading along the axis, whereas predominant longitudinal orientation results in better resistance to tensile stress. The predominant orientation of collagen within a cross-section of long bone is not random, but matches the expected distribution of mechanical stress across the section, and its rotational shift along the whole shaft. More transverse collagen is deposited at sites of compressive loading, and more longitudinal collagen is deposited at sites of tensile stress. These structural oriented bone tissues in a load-bearing bone are presumed to be laid down by the osteocytes present in the bone and bone remodeling mediates mechanical adaptation in compact bone.

A bone is typically comprised of bone tissue in the form of cortical and trabecular bone. Cortical bone is frequently referred to as compact bone and is the major load-bearing part of a bone. Trabecular bone is present in what is typically referred to as cancellous bone where it appears as densely interconnected structure of “spongy” bone. Spongy bone is typically bone that contains the hemotopoictic cellular elements which is called bone marrow. Trabecular bone can be described as forming a cross-bracing lattice between cortical bone in a bone. It is important to emphasize a need to differentiate between “a bone” and “bone”60 (as a tissue). A bone is comprised of bone tissue present as cortical and cancellous (spongy) bone.

The mineralized osteoid typical of bone tissue is hydrated along the organic molecular structure and is an essential element of the mineral structure. Hydrating molecules of water form complex molecular associations with these organic and non-organic elements of bone tissue and can be described as being tightly bound, loosely bound, and free. Free water and loosely bound water can frequently be removed from bone tissue with only minor changes in the overall mechanical characteristics of the bone tissue. Tightly bound water can be removed only under extreme conditions and results in significant changes in the physical and mechanical properties of bone tissue. In fresh bone, water serves a solvating function in bone tissue allowing proper orientation and molecular spacing of the collagen fibrils which maintain structural alignment of the mineral phase in association with the organic phase.

Bone tissue in the form of bone grafts for implantation into a patient, is typically preserved and provided in a dehydrated state. Dehydration of bone tissue through drying, whether by air drying or sublimation as in freeze-drying, results in alteration of the molecular structure of the bone tissue and as a result of the reorientation of the collagen fibrils and the crystalline mineral phase, stress accumulates in the bone tissue. This stress can be relieved by rehydration or by the occurrence of small or large dislocations of structure. Small dislocations are designated micro fractures and are not usually visible to the naked eye. Large dislocations are designated fractures and are usually visible to the naked eye.

In a long bone, for example a femur, tibia, fibula, or humerus, the shaft separates the proximal and distal ends of the long bone. The shaft serves to focus loads applied to the whole bone into a smaller diameter than found at the proximal and distal ends of the long bone and the shaft of a long bone is typically of a cylindrical shape and is comprised of compact (cortical) bone. Loads applied along the axis of the shaft require that the cortical bone maintains a constant circumference, i.e. the tendency to failure would distort the bone tissue perpendicular to the axis of load application. Thus, the orientation of the collagen fibers should be such that tensile stress is resisted along the axis of loading and compressive stress is resisted perpendicular to loading. Drying of shaft portions of long bones results in reorientation of collagen fibers and the mineral phase such that changes in the circumferential orientation create stress within the bone matrix which can be relieved only by rehydration or occurrence of a fracture which allows a reorientation approximating the original orientation. In dehydrated cortical ring grafts cut from the shafts of long bones, this stress release can present as a fracture along the long axis of the bone shaft leaving a circumference which approximates the circumference of the cortical ring graft prior to drying. By rehydrating bone grafts prior to implantation, the potential for fracture formation which can compromise the function of the bone product can be reduced, but not eliminated. Fractures as discussed above can occur in dehydrated bone prior to rehydration and result in a graft having compromised biomechanical properties, which in turn can result in graft failure when implanted in a patient.

Load-bearing soft tissue grafts such as ligaments, tendons, and fascia lata are frequently provided in a freeze-dried state. Such grafts must be rehydrated prior to clinical implantation. Such soft tissue grafts typically contain collagen, elastin, and assorted proteoglycans and mucopolysaccharides. The collagens and elastins are the load-bearing components of these soft tissue grafts and the assorted proteoglycans and polysaccharides serve to bind the fibrillar collagens into a matrix-like structure. The structural organization of fascia lata is similar to dura mater in being isotropic in load-bearing properties (Wolfinbarger, L., Zhang, Y., Adam, B L T., Hornsi, D., Gates, K., and Sutherland, V., 1994, “Biomechanical aspects on rehydrated freeze-dried human allograft dura mater tissues” J. Applied Biomaterials, 5:265-270) whereas tendons (for example the Achilles tendon) or ligaments (for example the Anterior cruciate ligament) are typically anisotropic in load-bearing properties. In these types of load-bearing soft tissue grafts, the tensile properties of the tissues depend on the flexibility of the collagenous structures to stretch under load and return to their original dimensions upon removal of the load.

A wide variety of bone and soft tissue products are used in veterinary, medical, orthopedic, dental, and cosmetic surgery applications. These bone and soft tissue products can be used in load-bearing and non-load bearing applications and the bone and soft tissue products can be supplied under a variety of forms. Bone products are provided as fresh-frozen, freeze-dried, rehydrated freeze-dried, air-dried, organic solvent preserved, or provided preserved by other similar types of preservation methods. Each method of preservation of bone product possesses selected advantages and disadvantages and thus the method of preservation is generally modified to select for specific needs of a given bone graft. Soft tissue products are typically provided as fresh-frozen or freeze-dried and each method of preservation of soft tissue products possess selected advantages and disadvantages and thus the method of preservation is generally modified to select for specific needs of a given soft tissue product.

Bone and soft tissue products preserved and stored by methods involving freeze-drying (removal of water by sublimation) yield a bone or soft tissue product which is significantly more brittle than normal bone or soft tissue, and has a tendency to fracture into numerous small pieces, which ultimately can result in graft failure. Specifically, freeze-drying causes grafts to be brittle and typically causes shrinkage where the shrinkage is often not uniform, thereby causing graft failure; solvent preservation using for example, acetone or alcohol, can cause irreversible denaturation of proteins, and solubilization of solvent soluble components, including for example, lipids. These alterations in materials properties of the bone and soft tissue products necessitates a rehydration step in preparation of the bone and soft tissue product for implantation. However, rehydration does not solve the problem that grafts can fracture prior to rehydration, thereby making rehydration futile, and if there are micro fractures prior to rehydration they remain after rehydration. These grafts are more likely to fail regardless of whether or not they are rehydrated. Even after rehydration the materials properties do not approximate the materials properties of normal bone.

Bone and soft tissue products are generally separated into load-bearing and non-load-bearing products. Examples of non-load bearing bone products are ground demineralized bone which are used for inducing new bone formation in a particular implant site. Load-bearing bone products are rarely demineralized and are used at implant sites where the bone graft will be expected to withstand some level of physical loads. It is therefore important that load bearing bone products not fail during implantation or during normal movements of the implant recipient and that the bone products not stimulate a pronounced physiological response. The majority of bone products are provided in either the fresh-frozen or freeze-dried format. The fresh-frozen format is undesirable because it includes donor derived bone marrow and is thus immunogenic and a source of disease transmission. The freeze-dried format is less of a problem than fresh-frozen grafts in the potential for disease transmission, however a freeze-dried bone graft is significantly more brittle than normal bone, more brittle than fresh-frozen bone, and must be rehydrated prior to clinical usage.

In that clinicians typically do not have time to adequately rehydrate bone graft products in the operating room, it is advantageous to provide a plasticized bone product which is less likely to fracture during insertion, does not need rehydration, possesses adequate materials properties, and is not a potential source for disease transmission.

### SUMMARY OF THE INVENTION

The present invention is based on the finding that plasticization of tissue or organ graft prior to implantation stabilizes and protects the tissue. As the plasticizer impregnates the graft during the plasticization process, the plasticizer replaces the water in the tissue or organ. Thus, plasticization prevents the tissue or organ from fracturing during storage and allows the tissue to be stored at room temperature or at a temperature lower than room temperature. Plasticization also helps maintain the tissue or organ so that it has similar materials properties as fresh or hydrated tissue or treated tissue. Plasticization also keeps the graft clean and free from infectious agents. Accordingly, plasticization allows for direct implantation of the graft into the subject without further processing or with minimal processing after removal from the packaging.

The present invention provides a plasticized tissue or organ graft suitable for transplantation comprising one or more plasticizers. The tissue may be soft tissue, hard tissue, or any tissue that can be plasticized. The tissue may be selected from the group consisting of bone, cartilage, heart valve, venous valve, blood vessel, ureter, dermis, small intestine, large intestine, periosteum, nerve, menisci, cartilage, non-valve patch, muscle, dura, pericardium, fascia, tendon, and ligament. The organ may be heart or skin.

The tissue or organ graft that is plasticized for transplantation may be from an allogenic source, an autogenic source, or a xenogenic source. The graft also may be derived from a mammal. The mammal may be a human, a cow, a pig, a goat, a dog, a horse, or a sheep.

The present invention provides an implantable, non-demineralized, load-bearing hard tissue products which are mechanically stabilized by use of biocompatible plasticizers.

The present invention provides an implantable, load-bearing, soft tissue products and organs which are mechanically stabilized by use of biocompatible plasticizers.

The present invention provides an implantable, load-bearing, hard tissue products which do not require rehydration.

The present invention provides an implantable, load-bearing, soft tissue products and organs which do not require rehydration.

The present invention provides methods of plasticizing load-bearing hard and soft tissue products.

The present invention provides plasticized hard and soft tissue products and organs which are resistant to proliferation of microorganisms.

The present invention provides hard and soft tissue products and organs which can be stored at room temperature using conventional packaging.

The present invention provides plasticized hard and soft tissue products and organs where the plasticizer can be readily removed prior to implantation.

The present invention provides plasticizers to plasticize hard and soft tissue products and organs which are not toxic to a recipient of the plasticized bone or soft tissue graft.

The present invention provides an implantable load-bearing hard and soft tissue products and organs which are similar in physical, chemical, and biological properties as compared to normal tissue (fresh bone or fresh soft tissues) or organ yet lack the inherent disadvantages (including for example, potential disease transmission, increased immunogenicity, and a tissue (e.g. bone marrow) which can yield toxic degradation products and/or retard graft incorporation) of fresh-frozen, dehydrated, and freeze-dried bone and/or soft tissue products.

The present invention provides a plasticized tissue graft or plasticized organ suitable for transplantation into a human, including a non-demineralized bone graft having an internal matrix essentially free from bone marrow elements; and one or more plasticizers contained in the internal matrix.

The present invention provides a plasticized tissue graft or organ, including a cleaned, non-demineralized, bone graft; and one or more plasticizers, where the cleaned non-demineralized bone graft is impregnated with the one or more plasticizers.

The present invention provides a plasticized tissue graft or organ, including a cleaned, non-demineralized, bone graft including one or more plasticizers. It is a further objective of the invention to provide a plasticized tissue graft or organ that can be sterilized using irradiation.

Plasticity of tissue or organ grafts depends primarily on the waters of hydration present in the matrix structure, where water movement under a load is restricted by the viscous nature of the proteoglycan/polysaccharide component, and bound waters of hydration in the collagen component affect the flexibility of the tensile component of the tissues. The invention deals with the plasticization of these load-bearing tissue products or organs where the water is replaced with one or more plasticizers including for example, glycerol (glycerin USP) (liquid substitution) such that the tissue or organ does not need to be rehydrated to remove the plasticizer prior to clinical implantation.

The present invention provides a plasticized hard or soft tissue product or organ, which requires no or minimal processing just prior to implantation, by providing a plasticized bone and/or soft tissue product that exhibits materials properties that approximate those properties present in normal hydrated tissue, is not brittle and does not necessitate rehydration prior to implantation.

The present invention provides a method for producing a plasticized tissue graft or plasticized organ suitable for transplantation into a human, by impregnating a cleaned, tissue graft or organ with one or more plasticizers to produce a plasticized tissue graft or plasticized organ. The plasticizer may be a polyol or a fatty acid. As an example, the plasticizer may be glycerol. The plasticizer composition may further comprise one or more biocompatible solvents. The biocompatible solvent may be an alcohol. The plasticizer composition may further comprise a permeation enhancer.

The conditions for plasticization will depend upon the tissue or organ being plasticized. Different tissues or organs will require different concentrations or percentages of plasticizer, different lengths of time of time for incubation, different temperatures for plasticization.

The present invention may employ a plasticizer composition comprising between about 10% v/v to 20% v/v plasticizer or about 15% v/v plasticizer for plasticizing skin or dermis.

The present invention may employ a plasticizer composition comprising greater than about 70% v/v plasticizer, between about 75% v/v to 85% v/v plasticizer, or about 80% v/v plasticizer for plasticizing vein, artery, or non-valve patch.

The present invention may employ a plasticizer composition comprising greater than about 70% v/v plasticizer, greater than about 75% v/v plasticizer, between about 75% v/v to 80% v/v plasticizer, or about 77% v/v plasticizer for plasticizing bone.

The present invention may also comprise cleaning and/or treating the tissue or organ graft prior to plasticization, although plasticization may also take place during the cleaning or treatment process. The graft may be cleaned to remove infectious and/or contaminating agents from it. The graft may be treated to enhance its performance and safety as a graft. Treating the tissue or organ graft may comprise treating the graft with modifying compositions, crosslinking, decellularizing, or devitalizing.

The modifying composition may comprise one or more growth factors, antithrombics, analigesics, antibiotics, cell adhesion molecules, chemotactants, or combinations thereof.

Crosslinking may comprise photooxidation or treatment with a crosslinking agent, such as but not limited to, formaldeyhyde, a dialdehyde, a diamine, and an epoxide. A dialdehyde may be a glutaraldehyde, malonaldehyde, glyoxal, succinaldehyde, adipalaldehyde, phthalaldehyde, or a derivative thereof.

Optionally, the plasticized tissue or organ graft may be dehydrated and/or sterilized. The plasticized graft may also be packaged for storage. The plasticized graft may be sterilized after packaging.

The present invention also provides a method of transplanting a tissue or organ graft into a subject comprising surgically implanting a plasticized tissue into a subject in need thereof. The graft may be derived from an allogenic source, an autogenic source, or a xenogenic source.

The present invention provides seeding a plasticized tissue prior to transplantation. The method comprising incubating a plasticized tissue with a medium; conditioning the tissue in the medium for cell seeding; seeding the cells onto a graft; and implanting the graft into a subject in need thereof.

Certain embodiments of the invention provide a method of preventing a tissue or organ graft for implantation from fracturing comprising incubating the graft with a plasticizer composition comprising one or more plasticizers under conditions that allow the one or more plasticizers to impregnate the tissue or organ. The plasticized graft may be sterilized and/or packaged for storage until implantation.

Other embodiments of the invention provide a method to enable storage of tissue or organ graft at room or low temperature comprising incubating the graft with a plasticizer composition comprising one or more plasticizers under conditions that allow the one or more plasticizers to impregnate the graft. The low temperature may be about −60° C. to −80° C. for storage of certain tissues, such as cardiovascular grafts.

The invention also provides a method of stabilizing tissue or organ graft in a dehydrated state comprising incubating a dehydrated graft with a plasticizer composition comprising one or more plasticizers under conditions that allow the one or more plasticizers to impregnate the graft.

### DETAILED DESCRIPTION

The below definitions serve to provide a clear and consistent understanding of the specification and claims, including the scope to given such terms

Alcohol. By the term “alcohol” is intended for the purposes of the present invention, one of a series of organic chemical compounds in which a hydrogen attached to carbon is replaced by a hydroxyl. Suitable alcohols useful in the plasticizer composition of the present invention preferably include C₁-C₁₀ alcohols, and more preferably ethanol and isopropyl alcohol.

Biocompatible. By the term “biocompatible” is intended for the purposes of the present invention, any material which does not provoke an adverse response in a patient. For example, a suitable biocompatible material when introduced into a patient does not itself provoke a significant immune response, and is not toxic to the patient.

Biomechanical strength. By the term “biomechanical strength” is intended for the purposes of the present invention, those properties exhibited by a tissue graft, including loading strength, compressive strength, and tensile strength.

Bone graft. By the term “bone graft” is intended for the purposes of the present invention, any bone or piece thereof or combination of pieces, obtained from a donor for example a human or animal and/or cadaver donor, including for example cortical bone and/or cancellous bone and/or cortico-cancellous bone, and including for example any essentially intact bone graft including for example any essentially intact bone graft including for example the femur, tibia, ilia, humorous, radius, ulna, ribs, whole vertebrae, mandibula and/or any bone which can be retrieved from a donor with minimal cutting of that bone for example, one half of an ulna, a femur cut in half to yield a proximal half and a distal half, femoral head, acetabula, distal femur, femur shaft, hemi-pelvi, humerus shaft, proximal femur, proximal femur with head, proximal humeri, proximal tibia, proximal tibi/plateaus, talus, tibia shaft, humeral head, ribs, and/or at least a substantial portion of a whole bone, i.e. at least one-quarter of a whole bone; and/or any cut bone grafts including for example an iliac crest wedge, a Cloward dowel, a cancellous cube, a fibular strut, cancellous block, a crock dowel, femoral candles, femoral ring, femur segment, fibula segment, fibular wedge, tibia wafer, ilium strip, Midas Rex dowel, tibial segment, and radius/ulna wedge; and/or a composite bone graft including cortical and/or cancellous and/or cortico-cancellous bone, and any combination thereof. The term may also include composite grafts.

Bone marrow elements. By the term “bone marrow elements” is intended for the purposes of the present invention, the highly cellular hematopoietic connective tissue filling the medullary cavities and spongy epiphysis of bones which may harbor bacterial and/or viral particles and/or fungal particles, and includes for example, blood and lipid.

Cleaned bone graft. By the term “cleaned bone graft” is intended for the purposes of the present invention, a bone graft that has been processed for example, using means know in the art, to remove bone marrow elements.

Dehydrated bone or soft tissue. By the term “dehydrated bone or soft tissue” is intended bone tissue or soft tissue which is preserved by dehydration, where dehydration includes replacement of water by a plasticizer and/or removal of water from a tissue by one or more drying methods including for example, freeze-drying, and/or sublimation and/or air drying and/or liquid substitution.

Essentially free from. By the term “essentially free from” is intended for the purposes of the present invention, a bone graft where the material removed (i.e., bone marrow elements) from the bone graft may not be detectable using detection means known in the art at the time of filing of this application.

Incubating. By the term “incubating” is intended for the purposes of the present invention, processing a bone graft in for example, a plasticizer composition by soaking the graft in the composition, shaking the graft with the composition, subjecting the graft to flow of the composition where the flow may be induced by negative or positive pressure, subjecting the graft and/or the composition to negative or positive pressure, soaking the bone graft in a plasticizer composition in a negative pressure environment, sonicating the graft with one or more plasticizer compositions, or centrifuging the graft with one or more plasticizer composition.

Impregnating. By the term “impregnating” is intended for the purposes of the present invention, any processing conditions which result in filling the internal matrix of a bone graft with a plasticizer composition.

Internal matrix. By the term “internal matrix” is intended for the purposes of the present invention, the spongy epiphysis of bones, the intercellular substance of bone tissue including collagen fibers and inorganic bone salts; or in soft tissue, the intercellular substance of such soft tissue including for example ligaments and tendons, including collagen and elastin fibers and base matrix substances.

Load-bearing. By the term “load-bearing” is intended for the purposes of the present invention a non-demineralized bone product or soft tissue product for implantation in a patient at a site where the bone graft or soft tissue graft will be expected to withstand some level of physical loads.

Materials properties. By the term “materials properties” is intended for the purposes of the present invention, those properties present in normal fresh bone which include for example, loading strength, compressive strength, tensile strength, and deformability.

Negative pressure. By the term “negative pressure” is intended for the purposes of the present invention, a pressure below atmospheric pressure, i.e. below 1 atm.

Natural or normal tissue or organ. By the term “normal tissue or organ” or “natural tissue or organ” is intended for the purposes of the present invention, fresh hydrated autogenous and/or fresh-frozen hydrated tissue or organ graft including for example, bone, fascia lata, ligaments, tendons, or skin.

Permeation or penetration enhancer. By the term “permeation enhancer” or “penetration enhancer” is intended for the purposes of the present invention, any agent including for example, isopropyl alcohol, surfactants, detergents and alcohols, that facilitates penetration of the one or more plasticizers or plasticizer composition into the bone or soft tissue. Permeation may be enhanced due to the reduced surface tension of the aqueous solution.

Plasticization. By the term “plasticization” is intended for the purposes of the present invention, replacing free and loosely bound waters of hydration in a tissue with one or more plasticizers without altering the orientation of the collagen fibers and associated mineral phase.

Plasticizer. By the term “plasticizer” is intended for the purposes of the present invention, any biocompatible compounds which are soluble in water and can easily displace/replace water at the molecular level and preferably have a low molecular weight such that the plasticizer fits into the spaces available to water within the hydrated molecular structure of the bone or soft tissue. Such plasticizers are preferably not toxic to the cellular elements of tissue into which the graft is to be placed, or alternatively, the plasticizer is easily removed from the graft product prior to implantation. Suitable plasticizers are preferably compatible with and preferably readily associate with the molecular elements of the bone tissue and/or soft tissue. Suitable plasticizers include for example: glycerol (glycerin USP), adonitol, sorbitol, ribitol, galactitol, D-galactose, 1,3-dihydroxypropanol, ethylene glycol, triethylene glycol, propylene glycol, glucose, sucrose, mannitol, xylitol, meso-erythritol, adipic acid, proline, hydroxyproline or similar water-soluble small molecular weight solutes which can be expected to replace water in the base matrix structure of bone tissue and/or soft tissue and provide the hydrating functions of water in that tissue. Suitable solvents include for example water, alcohols, including for example ethanol and isopropyl alcohol.

Plasticizer composition. By the term “plasticizer composition” is intended for the purposes of the present invention, any composition which includes one or more plasticizers and one or more biocompatible solvents. Suitable solvents include for example: water, and alcohols, including for example: C₁-C₁₀ alcohols, and more preferably ethanol and isopropyl alcohol.

Positive pressure. By the term “positive pressure” is intended for the purposes of the present invention, a pressure above atmospheric pressure, i.e. above 1 atm.

Rehydration. By the term “rehydration” is intended for the purposes of the present invention, hydrating a dehydrated plasticized tissue graft or a dehydrated non-plasticized tissue graft, with water, for example, prior to implantation into a patient. In the case of a plasticized graft, the plasticizer may optionally be not replaced by water or may optionally be partially or fully replaced by water.

Soft tissue or soft tissue grafts. By the term “soft tissue” or “soft tissue grafts” is intended for the purposes of the present invention, soft tissue exclusive of bone, including but not limited to connective tissue including ligaments and tendons, skin, dermis, vascular tissue including arteries and veins, pericardium, dura mater, fascia, heart valves, urethra, umbilical cord, and nerves. Other examples of soft tissues include heart valve, venous valve, blood vessel, ureter, skin, dermis small intestine, large intestines, periosteum, menisci, and muscle. Soft tissue grafts are composed of an internal matrix which includes collagen, elastin and high molecular weight solutes where during cleaning cellular elements and small molecular weight solutes are removed.

Hard tissue or hard tissue grafts. By the term “hard tissue” or “hard tissue grafts” is intended for the purposes of the present invention, tissue that has become mineralized or tissue having a firm intercellular substance. Examples of hard tissue include but are not limited to cartilage and bone.

Tissue or tissue grafts. By the term “tissue” or “tissue graft” is intended for the purposes of the present invention, hard and soft tissue that may be plasticized. Accordingly, the term includes various hard and soft tissues, such as but not limited to bone, cartilage, dermis, heart valve, venous valve, blood vessel, ureter, small intestine, large intestines, periosteum, menisci, cartilage, and muscle. The tissue may be from a natural source, synthetic source, or a combination thereof. The term “tissue” or “tissue graft” includes composite grafts such as composite bone graft.

The present invention provides biological tissues or organs plasticized by the methods of the present invention. Biological tissues or organs may include any tissue or organ that could be plasticized and that may be appropriate for implanting into animals, for example mammals and human beings. The tissues or organs may be from a mammalian source. The tissues or organs could be human or non-human such as bovine, porcine, canine, equine, ovine, or non-human primate, in origin. The tissues or organs may be derived from allogenic, autogenic, or xenogenic sources. Alternatively, the tissues or organs may be made from synthetic material or a combination of synthetic and natural material. The tissue or organ may include composite grafts, for example composite bone graft.

The organs may include liver, pancreas, heart, trachea, bladder, kidney, and skin. The tissues may be hard or soft and may be load-bearing or non-load-bearing. For instance, such tissues may include partial organs, bone, cartilage, peritoneum, pericardium, blood cells, heart valve leaflets, heart valves, aortic roots, aortic walls, pulmonary valves, pulmonary conduits, non-valved conduits, mitral valves, monocusps, tendons, ligaments, fascia, large and small vascular conduits, blood vessels, arteries, veins, diaphragm, pericardium, umbilical cords, dura mater, nerve, dermis, or tympanic membranes.

Some embodiments of the invention provide plasticized hard tissues such as but not limited to bone and cartilage. The bone may be demineralized or non-demineralized. Other embodiments of the invention provide plasticized soft tissues such as, but not limited to, heart valve, venous valve, blood vessel, ureter, dermis, small intestine, large intestine, periosteum, nerve, menisci, muscle, dura, pericardium, fascia lata, tendons, non-valve patch, and ligaments. Other embodiments of the invention provide plasticized organs such as, but not limited to, skin and heart.

Tissue and organ grafts may be cleaned and processed prior to plasticization. Plasticizers may also be added at various stages of cleaning and processing, such that the grafts may be plasticized during the cleaning and processing.

Various methods may be employed to clean and process tissues and organs grafts. The method used to clean and process the graft will depend on the type of tissue or organ. The plasticizers may be incorporated into the processing procedures using steps where the plasticizers are present at concentrations up to and including 100% v/v with or without permeation enhancers.

As an example, bone tissue may be cleaned and processed by transection of an essentially intact bone or perforation of an essentially intact bone with attachment of sterile plastic tubing to the cut end of a transected bone or to an attachment port inserted into the perforation of the perforated bone. The bone may be immersed in a cleaning solution, such solutions including known cleansing agents as well as those described in the above-referenced patents, with or without the use of for example sonication and/or centrifugation. The cleaning solution may be induced to flow into, through, and out of the bone through use of a peristaltic pump or negative pressure applied to the cleaning solution with or without the use of tubing. The induced flow of cleaning solution draws the bone marrow from the interior of the bone, and particularly from the cancellous bone marrow space, where it can be safely deposited in a receiving container containing a strong virucidal agent such as sodium hypochlorite (common bleach). The cleaned bone can then be further cleaned by causing the cleaning solution to be replaced with a solution of one or more decontaminating agents, including for example 3% hydrogen peroxide, with or without plasticizer. Hydrogen peroxide which in addition to its mild disinfection activity generates oxygen bubbles that can further assist in dislodging residual bone marrow materials causing the residual bone marrow material to flow from the bone and into the receiving container. The bone after cleaning and disinfection may be essentially free of infectious agents such as bacteria, viruses, viral particles, or fungi.

Soft tissues may be cleaned and processed by rinsing with Allowash® Solution or with an antibiotic solution. For example, tendon and ligament may be processed with Allowash® Solution, while cardiovascular grafts, such as patch valve and vein may be rinsed with a solution containing antibiotics.

After cleaning and processing but prior to plasticization, the graft may be dehydrated by freeze drying or lyophilization. The graft may also remain fully hydrated for plasticization.

In the above-described process, after processing with the cleaning solution, after processing with a decontaminating agent, in place of processing with a decontaminating agent, or after dehydration, the cleaned graft may be plasticized for example, by processing the cleaned graft with a plasticizer composition containing one or more plasticizers including for example glycerin USP in a solvent.

It is often necessary or desirable to treat a natural or synthetic tissue or organ graft to improve its performance and/or safety as a graft. The present invention provides processes for treating tissues or organs prior to or after plasticization to stabilize the tissues or organs from degradation, to reduce calcification, to reduce antigenicity, to improve its long-term durability and function, and/or to enhance its recellularization post implantation. The tissues or organs may be treated before, after, or during cleaning and disinfection. Any organ or tissue, hard or soft, may be treated to improve its efficacy and/or safety as a graft.

Various processes may be used to treat the different biological tissues or organs to improve their performance as a graft. The processes may involve treating the tissue or organ grafts with modifying compositions such as but not limited to antimicrobial compositions, anti-calcification compositions, fixation compositions, decellularization compositions, devitalization compositions, and compositions comprising bioactive factors, antithrombics, analgesics, antibiotics, antimicrobials, cell adhesion molecules, chemotactants, or combinations thereof. Treating the graft with modifying compositions and/or by various processes may alter host responses with respect to healing, immunogenicity, and thrombogenicity. Treatment fluids may also include a solvent, buffer, inert salts and other excipients that do not significantly alter the tissue or organ.

Compositions having antimicrobial activity may include compositions comprising antibiotics, for example, penicillin, vancomycin, minocycline, rifampicine, streptomycin, amphotericin, licomycin, cephalosporin, and polymyxin. Some metal cations have antimicrobial activity. Such metal cations include but are not limited to ions of Ag, Au, Pt, Pd, Ir, Cu, Sn, Sb, Bi and Zn. Accordingly, some compositions may include metal cations for inhibiting microbial activity. Compositions comprising bioactive factors may include compositions comprising one or more bioactive factors such as an osteogenic factor, a chondrogenic factor, a cytokine, a mitogenic factor, a chemotactic factor, a cementum attachment protein (CAP), a transforming growth factor (TGF), a fibroblast growth factor (FGF), an angiogenic factor, an insulin-like growth factor (IGF), a platelet-derived growth factors (PDGF), an epidermal growth factor (EGF), a vascular endothelial growth factor (VEGF), a nerve growth factor (NGF), a neurotrophin, a bone morphogenetic protein (BMP), osteogenin, osteopontin, osteocalcin, cementum attachment protein, erythropoietin, thrombopoietin, tumor necrosis factor (TNF), an interferon, a colony stimulating factor (CSF), or an interleukin, among others.

Some metal cations, such as polyvalent metal cations, shown to inhibit calcification. Examples of these polyvalent metal cations include but are not limited to Al⁺³, Mg⁺², and Fe⁺³. Other calcification inhibitors include alcohols, toluidine blue, diphosphates, and detergents such as sodium dodecyl sulfate, and alpha amino oleic acid. Thus, one or more polyvalent metal cations or calcification inhibitors may be added to the composition to inhibit calcification.

Other ways to reduce calcification include fixation of the tissue or organ grafts and decellularization of the grafts. Fixation of the tissue or organ graft stabilizes the graft by preventing enzymatic degradation. The grafts may be fixed by chemical crosslinking with various agents having two or more functional groups. Crosslinking agents include diamines, epoxides, dialdehydes, carbodiimides, diisocyanates, and formaldehyde, which is capable of acting difunctionally. Dialdehydes include, for example, glutaraldehyde, malonaldehyde, glyoxal, succinaldehyde, adipalaldehyde, phthalaldehyde and derivatives thereof. Derivatives of glutaraldehyde include, for example, 3-methylglutaraldehyde and 3-methoxy-2,4-dimethyl glutaraldehyde. It is noted that formaldehyde may not always function as a satisfactory crosslinking agent, but formaldehyde is commonly used as a sterilant to store grafts following glutaraldehyde crosslinking. Ethanol is also commonly used as a sterilant for cross-linked grafts.

Tissues and organs may also be crosslinked by photooxidation. Photooxidation involving ultraviolet (UV) and gamma irradiation, dehydrothermal treatment, and photosensitive dye have been employed. Photosensitive dyes used in photooxidation include methylene blue, methylene green, rose bengal, riboflavin, proflavin, fluorescein, rosin, and pyridoxal-5-phosphate. These dyes when activated are believed to cause a transfer of electrons or hydrogen atoms, and thereby oxidize a substrate if oxygen is present.

Prior to or post fixation of the tissue or organ graft with a fixative agent such as formaldehyde or glutaraldehyde, the tissue may be immersed in a solution containing a denaturant, surfactant, and crosslinking agent at a temperature between about 4° C. to about 50° C., or about 20° C. to about 30° C. for a period about 1 h our to 36 hours. Denaturants include but are not limited to alcohols/solvents (ethanol, acetones, ethers of small alkyl size), acidified ethers (sulfuric acid/ether mixture), ketones (methyl ethyl ketone), chlorofluorocarbon solvents, glycols (glycerol ethylene glycol and polyethylene glycols), chaotropic agents (urea, guanidine hydrochloride, guanidine thiocyanate potassium iodide), and high concentration salt solutions (lithium chloride, sodium chloride, and cesium chloride). Surfactants include but are not limited to anionic surfactants (sodium dodecyl sulfate), alkyl sulfonic acid salts (1-decanesulfonic acid sodium salt), non-ionic compounds (Triton X-100), and alkylated phenoxypolyethoxy alcohol (NP40, Tween 80, Tween 20, octyl-derivatives, CHAPS, and CHAPSO). Crosslinking agents include but are not limited to aldehydes, expoxides, carbodiimides, and diisocyanates.

Because nonviable cells in transplant tissues or organ are sited for calcium deposition, various processes have been developed for decellularization of tissues or organ prior to transplantation. Some decellularization processes are based on detergent treatments. Detergents that may be used for decellularization include but are not limited Triton-X, polyoxyethylene (20) sorbitan mono-oleate and polyoxyethylene (80) sorbitan mono-oleate (Tween 20 and 80), sodium deoxycholate, 3-[(3-chloramidopropyl)-dimethylammino]-1-propane-sulfonate, octyl-glucoside, and sodium dodecyl sulfate. As an example, soft tissues may be decellularized by subjecting the tissues to an induced pressure mediated flow of an extracting solution, followed by inducing a pressure mediated flow of a treating solution, then washing the treated tissue to produce the acellular graft. The extracting solution may comprise a nonionic detergent such as polyoxyethylene alcohol, polyoxyethylene isoalcohol, polyoxyethylene p-t-octylphenol, polyoxyethylene nonylphenol, polyoxyethylene esters of fatty acids, and polyoxyetylene sorbitol esters. The treating solution may comprise sodium dodecylsulphate, sodium dodecylsulphonate, sodium dodecyl-N-sarcosinate, and sodium suramin. Decellularization processes also may be accomplished using enzymes, such as dispase II, trypsin, chymotrypsin, and thermolysin, and salts.

The tissues or organ may also be devitalized prior to plasticization, so as to retain metabolically non-viable and/or reproductively non-viable cells, and large molecular weight cytoplasmic proteins. Devitalized tissues or organ have improved long-term durability and function, and enhanced recellularization post-implantation.

Devitalization processes may be based on detergents. The detergent may be a nonionic detergent, such as but not limited to N-lauroylsarcosinate, a polyoxyethylene alcohol, a polyoxyethylene isoalcohol, a polyoxyethylene p-t-octyl phenol, a polyoxyethylene nonyphenol, a polyoxyethylene ester of a fatty acid, and a polyoxyethylene sorbitol ester. As an example, the devitalization process may involve subjecting the tissue to an induced pressure mediated flow of an extracting solution, optionally followed by inducing a pressure mediated flow of a salt solution, then washing the tissue to produce the devitalized graft. The extracting solution may have N-lauroylsarcosinate. The extracting solution may or may not contain a denaturing agent.

In addition to the detergents, protease inhibitors may be added to the processing solutions of the decellularization and the devitalization process to prevent degradation of the extracellular matrix. Examples of protease inhibitors that are useful include but are not limited to N-ethylmaleimide (NEM), phenylmethylsulfonylfluoride (PMSF) ethylenediamine tetraacetic acid (EDTA), ethylene glycol-bis-(2-aminoethyl(ether)NNN′N′-tetraacetic acid, ammonium chloride, elevated pH, apoprotinin, and leupeptin. Endonucleases capable of degrading both deoxyribonucleic acids and ribonucleic acids, as well as decontaminating agents may also be included in the processing solution. Examples of decontaminating agents include antimicrobial agents, alcohols, chlorine dioxide, and antibiotics.

Optimum treatment conditions including concentration of agents, length of time of incubation with agents, treatment or extraction solutions, and temperature may be employed. The conditions will vary depending on the type of tissue or organ, the agent, and/or the treatment process,

After treatment, the tissues or organ graft may be dehydrated by freeze drying or lyophilization. Alternatively, after treatment, the graft may be fully hydrated. Plasticizers may be introduced into freeze dried or fully hydrated crosslinked tissues or organs. Plasticizers also may be introduced into freeze dried or fully hydrated non-crosslinked grafts.

As an example, tissue or organ grafts may be incubated with a modifying composition comprising one or more growth factors to enhance its efficacy, stability, and/or safety as a graft after implantation. For example, dermis, cardiovascular tissues, pericardium, and fascia may be treated with a modifying composition to improve their performance and/or safety as a graft. After incubation with a modifying composition to enhance its efficacy in a subject, the grafts may be stored with a plasticization composition. Hard tissues such as bone may be treated with Allowash® Solution or modified with growth factors, cell adhesion molecules, and chemotactants. Cartilage may be treated with a modifying composition or devitalized. Collagen based tissue grafts may be treated by heat cross-linking.

Plasticizers may be introduced into the biological tissue or organ grafts at any of several points in the cleaning and processing and treatment procedures. Plasticization of grafts represents a method of replacing free and loosely bound waters of hydration in the graft with a plasticizer composition containing one or more plasticizers, without altering the orientation of the collagen fibers and associated mineral phase. Suitable plasticizers include compounds which are soluble in water and can displace/replace water at the molecular level. Suitable plasticizers preferably have a low molecular weight such that the plasticizer fits into the spaces available to water within the hydrated molecular structure of the bone or soft tissue. Such plasticizers are not toxic to the cellular elements of the tissue or organ into which the graft is to be placed, or alternatively, the plasticizer is easily removed from the graft product prior to implantation. Finally, the plasticizer is preferably compatible with and preferably readily associates with the molecular elements of the bone or soft tissue.

Plasticizers suitable for use in the present invention include for example, a variety of biocompatible aqueous solutions. Examples of acceptable plasticizers include, but are not restricted to, members of the polyol family (sugar alcohols) of compounds including C₂-C₇ polyols, monoglycerides (such as mono-olein and mono-linolein), and various short- and medium-chain free fatty acids (such short-chain free fatty acids preferably having a carbon chain length of less than six (<C₆); and such medium-chain free fatty acids preferably having a carbon chain length of from C₁₂-C₁₄) and their corresponding monacylglycerol esters (Mgs) such as the saturated Mgs, ranging in carbon chain length C₅-C₁₆, and preferably C₅-C₁₄ Mgs. Specific plasticizers include, but are not limited to, glycerol (glycerin USP), glycerolmonolaurate, adonitol, sorbitolt, ribitol, galactitol, D-galactose, 1,3-dihydroxypropanol, ethylene glycol, triethylene glycol, propylene glycol, glucose, sucrose, mannitol, xylitol, meso-erythritol, adipic acid, proline, hydroxyproline or similar water-soluble small molecular weight solutes which can be expected to replace water in the matrix structure of bone, soft tissue, or organ, and provide the hydrating functions of water in that tissue or organ. Other plasticizers suitable for use in the present invention can be readily selected and employed by one of ordinary skill in the art to which the present invention pertains without undue experimentation depending on the desired clinical outcome, sensitivity of the implantation procedure, patient sensitivities, and physician choice.

The present plasticizers may be employed at concentrations in the range of from about 10% to 100% by weight or volume. Depending on the tissue or organ being plasticized, the present plasticizers may be employed at concentrations in the range of from about 13% v/v to about 100% v/v, from about 50% v/v to about 100% v/v, from about 60% v/v to about 100% v/v, from about 75% v/v to about 100% v/v, from about 10% v/v to about 25% v/v, from about 10% v/v to about 20% v/v, from about 75% v/v to about 90% v/v, from about 75% v/v to about 85% v/v, from about 80% v/v to about 85% v/v, or from about 75% v/v to about 85% v/v of the total volume. As an example, the concentration of plasticizer used to plasticize skin or dermis may be from about 10% to about 25% v/v, from about 10% to about 20% v/v, or about 15% v/v; the concentration of plasticizer used to plasticize bone may be greater than about 70% v/v, greater than about 75% v/v, from about 70% to about 90% v/v, from about 75% to about 80% v/v, about 70% v/v, or about 77% v/v; and the concentration of plasticizer used to plasticize vein, artery, or non-valve patch may be greater than 70% v/v, from about 70% to about 90% v/v, from about 75% to about 85% v/v, or about 80% v/v.

One or more plasticizers may be introduced into the biological tissue or organ graft to plasticize the tissue. Alternatively, the graft may be plasticized with a composition comprising one more plasticizers or a composition comprising one or more plasticizers and one or more biocompatible solvents. Biocompatible solvents include but are not limited to water, saline, glucose solution, dextrose glucoside solution, lactated ringers solution, and alcohol. The biocompatible solvent may be a permeation enhancer that facilitates the penetration of the one or more plasticizers into the graft. The plasticization composition may comprise the plasticizer and no biocompatible solvent or the plasticization composition may consist essentially of the plasticizer.

The plasticizer may be introduced into the biological tissue matrix at any number of steps in the processing and treatment procedures and at a variety of concentrations with and/or without the use of permeation enhancers. The results of plasticization of tissues and organs are tissue products, such as bone or soft tissue products, and organs which are not subject to fractures or micro-fractures (as in the case of traditional dehydrated graft products), yet do not need to be rehydrated prior to use. Plasticization may prevent fractures from occurring in grafts and may reduce the propagation and elongation of fractures. For instance, some cortical bone may have existing small fractures. Sterilization by irradiation of such cortical bone may elongate or increase the number of fractures. Plasticization may prevent the existing fractures in the cortical bone from elongating or propagating during the sterilization process.

The mechanical and use properties of a plasticized bone, soft tissue, or organ product are similar to those of natural (fresh autogenous and/or fresh-frozen allograft) or treated bone, soft tissue, or organ. As an example, the graft may be fixed, plasticized, and stored in a closed package system with sterile interconnected solution containers. The tissue or organ may be fixed and sterilized in glutaraldehyde. Subsequently, the glutaraldehyde may be rinsed out and replaced with glycerol for plasticization. The plasticized tissue or organ may then be stored in glycerol. Eventually, the liquid may be removed from the graft package.

In some embodiments of the invention, the enhancers may be added to the tissue or organ prior to the plasticization process or prior to the addition of the plasticizer. In other embodiments, the enhancers may be added with the plasticizer to the tissue or organ. Various types of permeation or penetration enhancers may be added to the plasticization process to enhance penetration of the plasticizer into the biological tissue or organ. Permeation or penetration enhancers include, but are not limited to, chemical enhancers and solubility enhancers.

Chemical enhancers enhance molecular transport rates across tissues or membranes by a variety of mechanisms. Many different classes of chemical enhancers have been identified, including cationic, anionic, and nonionic surfactants (sodium dodecyl sulfate, polyoxamers); fatty acids and alcohols (ethanol, oleic acid, lauric acid, liposomes); anticholinergic agents (benzilonium bromide, oxyphenonium bromide); alkanones (n-heptane); amides (urea, N,N-diethyl-m-toluamide); fatty acid esters (n-butyrate); organic acids (citric acid); polyols (ethylene glycol, glycerol); sulfoxides (dimethylsulfoxide); and terpenes (cyclohexene).

Some fatty acids disrupt the lipid bilayer and enhance the permeability through lipids. Examples of lipid permeation enhancers include, but are not limited to, linoleic acid, capric acid, lauric acid, and neodecanoic acid. These lipids may be dissolved in a solvent such as ethanol or propylene glycol.

Another way to increase the permeation of the plasticizer may be to use solubility enhancers that increase the plasticizer's solubility in excipients that are added to the plasticizer composition. Solubility enhancers include water diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl)pyrrolidone, N-methylpyrrolidone, 1-dodecylazacycloheptan-2-one and other n-substituted-alkyl-azacycloalkyl-2-ones.

A combination of enhancers also may be added to the plasticizer composition to increase the penetration of the plasticizer into the tissue or organ graft. The permeation enhancer may be added to the plasticizer composition. The permeation enhancer may also be added prior to adding the plasticizer. For example, the permeation enhancer may be added during the processing step prior to plasticization or after the treatment step and prior to plasticization. The permeation enhancer may also be added during plasticization but prior to the addition of the plasticizer.

During the process of plasticization, the water in the tissue or organ graft may be replaced with one or more plasticizers including for example, glycerol (glycerin USP) (liquid substitutions such that the graft does not need to be rehydrated to remove the plasticizer prior to clinical implantation. Plasticization stabilizes and protects the graft so that the graft will not fracture during storage. Plasticization also maintains the graft in its clean stage, essentially free of infectious agent, and plasticization maintains the materials properties of the graft such that the graft has similar materials properties as fresh or hydrated tissue or organ or treated tissue or organ.

The present invention provides methods of plasticizing tissue or organ grafts for implantation. The grafts may be cleaned and processed and optionally treated prior to plasticization. Thus, depending on the type of tissue or organ, it may be fully hydrated or freeze dried when ready for plasticization. Prior to plasticization, the tissues or organs also may be treated with minimal cleaning to improve its performance as a graft. It is also part of the present invention to plasticize a graft and then freeze-dry it according to standard lyophilization/freeze-drying practices. Freeze-drying a tissue or organ graft that has been plasticized with, for example glycerol, permits the further “dehydration” of the tissue without any associated increase in brittleness because the glycerol will not leave the graft during the freeze-drying process, but rather will become more concentrated by the freeze-drying process and thus replace even more of the water during the dehydration process.

The grafts may be cleaned, processed, and/or treated using conventional methods or those described above. The grafts may be plasticized by adding one or more plasticizers or one or more plasticizer compositions to the processing steps after cleaning is essentially completed. The grafts may also be plasticized after the grafts have been treated, for example by crosslinking, decellularizing, or devitalization.

One or more plasticizers may be added to the tissue or organ grafts, and the plasticizers may be induced to penetrate into the grafts optionally using a permeation enhancer. The grafts may be fully hydrated prior to the plasticization process. Thus, the tissue is plasticized, yet the materials properties of the tissue is similar to the materials properties of normal tissue or tissue after cleaning, processing, and/or treatment to enhance its performance as a graft. For example, the plasticized bone will have the materials properties of hydrated bone, and plasticized soft tissue will have the materials properties of a hydrated soft tissue, and a plasticized crosslinked soft tissue will have the materials properties of a crosslinked soft tissue. Likewise, the plasticized organ will have the materials properties of normal organ or organ after cleaning, processing, and/or treatment to enhance its performance as graft.

Tissues or organs may be plasticized in a composition consisting essentially of plasticizer and no biocompatible solvent. The plasticizer composition may also be prepared by mixing the plasticizer with water, saline, aqueous solution, or an alcoholic solution. The plasticizer composition may comprise one or more permeation enhancers and other excipients.

Depending on the tissue or organ, various percentages of plasticizer, such as glycerol, and permeation enhancer, such as isopropanol, may be used. Isopropanol facilitates penetration of the glycerol into the tissue or organ by acting as a permeation enhancer, and glycerol more readily penetrates the tissue or organ due to the reduced surface tension of the alcoholic solution. The induced flow of glycerol/isopropyl alcohol into, through, and out of for example, the essentially intact bone, further serves to remove residual cellular elements, for example bone marrow materials, if any. It also allows penetration of the glycerol/isopropyl alcohol solution into the most remote areas of the tissue or organ, and facilitates a uniform distribution of the glycerol into the tissue or organ.

For instance, bone and soft tissue may be plasticized by processing with one or more plasticizer compositions containing one or more plasticizers, including for example, glycerin USP, in a solvent by for example drawing the plasticizer composition into the bone or soft tissue. Suitable solvents include for example isopropyl alcohol. The alcohol/plasticizer composition may be prepared by diluting absolute (100%) isopropyl alcohol with one or more plasticizers, including for example glycerin USP such that the plasticizer accounts for from about 10% to about 100% v/v, from about 50% to about 100% v/v, from about 60% to about 100% v/v, from about 75% to about 100% v/v, from about 15% to about 25% v/v, from about 15% to about 20% v/v, from about 75% to about 90% v/v, from about 75% to about 85% v/v, or from about 80% to about 85% v/v, of the total volume. The isopropyl alcohol accounts for from about 90% to about 0% v/v, from about 50% to about 0% v/v, from about 40% to about 0% v/v, from about 25% v/v to about 0% v/v, from about 85% to about 75% v/v, from about 85% to about 80% v/v, from about 25% to about 10% v/v, from about 25% to about 15% v/v, or from about 20% to about 15% of the total volume. As an example, the concentration of plasticizer used to plasticize skin or dermis may be from about 10% to about 25% v/v, from about 10% to about 20% v/v, or about 15% v/v; the concentration of plasticizer used to plasticize bone may be greater than about 70% v/v, greater than about 75% v/v, from about 70% to about 90% v/v, from about 75% to about 80% v/v, about 70% v/v, or about 77% v/v; and the concentration of plasticizer used to plasticize vein, artery, or non-valve patch may be greater than about 70% v/v, from about 70% to about 90% v/v, from about 75% to about 85% v/v, or about 80% v/v.

Some tissues or organ may require a higher or lower plasticizer concentration after processing or treatment. For example, a soft tissue, such as a fascia lata, pericardium, cardiovascular graft vein, or artery, may require less than about 80% v/v glycerol after crosslinking by photooxidation or with a chemical agent such as formaldehyde, a dialdehyde, a diamine, or an epoxide. Dialdehydes include, for example, glutaraldehyde, malonaldehyde, glyoxal, succinaldehyde, adipalaldehyde, phthalaldehyde and derivatives thereof. Derivatives of glutaraldehyde include, for example, 3-methylglutaraldehyde and 3-methoxy-2,4-dimethyl glutaraldehyde. Accordingly, the plasticizer concentration will be adjusted according to the processing and/or treatment steps used on the tissue or organ.

Certain conditions employed during the plasticization process will enhance penetration of the plasticizer into the tissue or organ graft. For example, permeation of the plasticization may be dependent on the temperature, the length of time of the incubation period, and incubation conditions. However, these conditions are also dependent on the type of tissue or organ and whether the tissue or organ was treated. Accordingly, various conditions for plasticization may be employed for the different grafts. Optimum conditions used during the plasticization process will improve the penetration of the plasticizer into the tissue or organ.

The plasticization process may be carried out at room or ambient temperature, for example about 20° C. to 28° C. However, the temperature may be increased or decreased depending on the type of tissue or organ and whether the tissue or organ has been treated. For example, it may be necessary to increase the temperature to about 35° C. to 55° C. or 37° C. to 44° C. For example, if the tissue is a soft tissue and has been treated by photooxidation or with one or more crosslinking agents, such as a diamine, an epoxide, a dialdehyde, or formaldehyde an increased temperature may be beneficial. Dialdehydes include, for example, glutaraldehyde malonaldehyde, glyoxal, succinaldehyde, adipalaldehyde, phthalaldehyde and derivatives thereof. Derivatives of glutaraldehyde include, for example, 3-methylglutaraldehyde and 3-methoxy-2,4-dimethyl glutaraldehyde. The temperature may also be decreased to a low temperature such as about 0° C. to 20° C., about 4° C. to 15° C., about 4° C. to 10° C., or about 4° C. to 6° C.

The length of time that the tissue or organ graft is exposed to the plasticizer may also enhance the permeation of the plasticizer into the tissue or organ. The graft may be incubated in the plasticizer composition for about 30 minutes to 30 hours, about 1 hour to 24 hours, about 2 to 12 hours, about 3 to 8 hours, or about 6 hours. As an example, a non-crosslinked non-valve patch may require about 24 to 30 hours of incubation with circulation of the plasticizer solution for penetration of the plasticizer into the tissue. A soft tissue that has been treated may require longer incubation time than an untreated tissue. For example, a soft tissues, such as a fascia lata, pericardium, cardiovascular graft, vein, or artery, that have been crosslinked by photooxidation or with one or more agents such as a diamine, a carbodiimide, a diisocyanate, an epoxide, a dialdehyde, or formaldehyde, prior to plasticization may require a longer incubation time than a soft tissue that has not been crosslinked. Dialdehydes include, for example, glutaraldehyde, malonaldehyde, glyoxal, succinaldehyde, adipalaldehyde, phthalaldehyde and derivatives thereof. Derivatives of glutaraldehyde include, for example, 3-methylglutaraldehyde and 3-methoxy-2,4-dimethyl glutaraldehyde. A crosslinked or fixed graft may require a few days of incubation and/or circulation of the plasticizer solution in order for the plasticizer to penetrate into the graft. For example, a treated tissue may require about two to ten days, about three to eight days, or about five to six days of incubation and/or circulation with plasticizer.

The graft may be immersed in a plasticizer composition under static condition. The graft also may be incubated in a plasticizer composition with agitation or shaking to help the plasticizer penetrate into the graft. Other methods used to enhance permeation of the plasticizer into the graft during incubation include applying centrifugal force or negative pressure by vacuum or by circulating the plasticizer solution through the graft. The graft may be centrifuged at a speed of from about 100 to 4,000 rcf about 500 to 2500 rcf, or about 1000 to 2000 rcf for a time of from about 10 minutes to 7 hours, about 30 minutes to 4 hours, or about 1 hour to 2 hours. A vacuum of about 10 to 50 mTorr, about 20 to 40 mTorr, or about 30 mTorr may be applied to the graft immersed in the plasticizer composition.

As an example, if desired, plasticized cancellous bone grafts may optionally be dry spun to remove any excess plasticizer present. Plasticized cortical bone grafts may optionally be blotted to remove any excess plasticizer present. The cleaned and plasticized grafts can then be packaged and stored at room temperature or under refrigeration. The plasticized grafts may be stored in plasticizer, such as glycerol, with minimal chemical residues.

Alternatively, tissue or organ grafts may be plasticized after cleaning and freeze-drying. For example, tissues or organs may be processed and cleaned according to any method including known methods, or as described above. After removal of bone marrow and/or cellular components, the grafts can be processed for example by freeze-drying. Freeze-dried or dehydrated grafts preferably contain less than about 5% residual moisture, satisfying the definition of freeze-dried bone allografts as prescribed under Standards of the American Association of Tissue Banks.

For instance, clean freeze-dried or dehydrated bone or soft tissue grafts are plasticized by processing the tissue graft with a plasticizer composition, suitable compositions including for example 20% isopropyl alcohol/80% glycerin USP or 100% glycerin USP. The alcohol/plasticizer composition can be prepared by diluting absolute (100%) isopropyl alcohol with the one or more plasticizers, including for example glycerin USP, such that the plasticizer accounts for from about 10% to 100% v/v, from about 60% to 100% v/v, or from about 10% to 20% v/v, from about 75% to 85% v/v, from about 75% to 80% v/v, of the total volume and isopropyl alcohol accounts for from about 90% to 0% v/v, from about 40% to 0% v/v, from about 90% to 80% v/v, or from about 25% to 15% v/v, of the total volume. As an example, the concentration of plasticizer used to plasticize skin or dermis may be from about 10% to about 25% v/v, from about 10% to about 20% v/v, or about 15% v/v; the concentration of plasticizer used to plasticize bone may be greater than 70% v/v, greater than 75% v/v, from about 70% to about 90% v/v, from about 75% to about 80% v/v, about 70% v/v, or about 77% v/v; and the concentration of plasticizer used to plasticize vein, artery, or non-valve patch may be from about 70% to about 90% v/v, from about 75% to about 85% v/v, or about 80% v/v.

Due to the presence of air in the cancellous and cortical bone spaces, the plasticizers may only penetrate into the bone tissue with which it is in physical contact. Suitable methods for achieving physical contact between the plasticizer and bone or soft tissue include those methods known to one of ordinary skill in the art to which the present invention pertains. The plasticizer composition can be induced to flow into the cancellous and cortical bone spaces of bone tissue, or soft tissue, thus achieving physical contact, by various known methods that can be readily selected and employed by one of ordinary skill in the art to which the present invention pertains without undue experimentation, and include for example, agitation of the tissue with the plasticizer composition, application of a vacuum (10 to 50 mBarr) above the plasticizer. Other methods of achieving physical contact include centrifuging the bone or soft tissue graft with the plasticizer composition, for example centrifugation at about 100 rcf (relative centrifugal force)—4,000 rcf, more preferably about 1000-4000 rcf more preferably about 1200 ref for about 30 min. to about 5 hrs or more. The vacuum induces the air trapped in the, for example, cancellous and cortical bone spaces/tissue to move out. The plasticizer quickly moves into the spaces previously occupied by air greatly enhancing penetration of the plasticizer into the bone or soft tissue. The plasticizer fills the spaces previously occupied by the free and bound water restoring the tissue to a materials property similar to that materials property of the original natural tissue.

The present one or more plasticizers may be introduced to tissues or organs at several points in the processing procedures. Plasticizers may be introduced prior to the freeze-drying or dehydrating step. By introducing plasticizers prior to freeze-drying or dehydrating, the derived plasticized soft tissue graft does not need to be freeze-dried, and can be packaged and stored at room temperature or refrigerated and can be implanted without rehydration reconstitution.

Prior to packaging, excess glycerol may optionally be removed from the plasticized bone or soft tissue graft, for example, by centrifugation. Specifically, in the case of cancellous bone grafts, the plasticized grafts are placed into a centrifuge vessel or container and on top of inserts designed to keep the bone grafts off of the bottom of the containers. The grafts are then centrifuged at 100-4,000 ref, preferably about 1200 rcf, for from about 10 sec. to 3 minutes, preferably 1 minute or less. The excess glycerol or similar plasticizer exits the grafts and collects in the bottom of the centrifuge containers away from the grafts.

The plasticized grafts may then be packaged directly or packaged in a packaging format which permits application of a vacuum to the container. The latter packaging format permits storage of grafts under vacuum and allows the ability to predict possible loss of sterility with loss of vacuum to the packaging. The grafts may be vacuum packed in peal-packs and stored at room temperature or under refrigeration.

The plasticized grafts may be sterilized prior to packaging or after packaging. The plasticized grafts may be sterilized, for example, by gamma, e-beam or other irradiation techniques, ethylene oxide gas, supercritical carbon dioxide gas, or any other suitable sterilization technique.

The plasticized tissue or organ grafts also may be sterilized by immersing in or incubating with a sterilant solution, such as glutaraldehyde or formaldehyde, and heated for a period of time sufficient to ensure sterility of the graft until implantation. The sterilization time and temperature may be about 1 to 7 days at 37° C. or 1 to 3 days at 50° C. The sterilant composition may contain about 0.2 to 2.5% by weight sterilant or about 0.25 to 1% by weight sterilant.

The method of the present invention allows the storage of the graft at room temperature or at low temperature and in dehydrated form after plasticization. For example, soft tissue grafts may be stored at 0° C. in dehydrated state and bone may be stored at room temperature. Some grafts, such as cardiovascular grafts, may be stored at −60° C. to −80° C. Plasticization stabilizes the tissue or organ graft after cleaning and treatment and thus, prevents the graft from fracturing during storage or prior to implantation. The plasticized tissue has similar materials properties as fresh or hydrated tissue, or treated tissue, and the plasticized organ has similar materials properties as fresh or hydrated organ, or treated organ. Plasticization enables storage of tissues at these cold temperatures in a “frozen” or “vitrified” state without damage normally associated with the formation of ice crystals in the tissues. Moreover, storage at low temperatures, such as −60° C. to −80° C., may impede the formation of toxic by products, such as aldehydes.

The method of the present invention produces plasticized tissues, such as bone and soft tissues, and plasticized organs containing minimal quantities of chemical residues and/or plasticizers. Accordingly, the plasticized tissue or organ may be removed from the package and directly implanted into a patient without rinsing to remove chemical residues or plasticizers. Also, the tissue or organ need not be rehydrated prior to implantation. If the presence of these small quantities of plasticizer is of concern, the plasticized tissue or organ may be quickly rinsed and/or washed in sterile saline or water just prior to implantation.

The present invention also provides for seeding cells onto a plasticized graft prior to implantation to improve the graft's ability to repair a bone defect or injury or to enhance fusion. As an example, prior to implantation, a plasticized graft may be incubated with cell culture medium to rinse off glycerol and pre-conditioned for cell seeding. Various cells, including stem cells and differentiated cells, may be seeded onto the plasticized graft. The cell seeded graft may then be transplanted into a subject.

Immediately prior to implantation, the plasticized graft may be treated with modifying compositions comprising various agents such as antibiotics, bioactive factors, antithrombics, cell adhesion molecules, chemoattractants, or combinations thereof to enhance its performance as a graft. The bioactive factor may be an osteogenic factor, a chondrogenic factor, a cytokine, a mitogenic factor, a chemotactic factor, a cementum attachment protein (CAP), a transforming growth factor (TGF), a fibroblast growth factor (FGF), an angiogenic factor, an insulin-like growth factor (TGF), a platelet-derived growth factors (PDGF), an epidermal growth factor (EGF), a vascular endothelial growth factor (VEGF), a nerve growth factor (NGF), a neurotrophin, a bone morphogenetic protein (BMP), osteogenin, osteopontin, osteocalcin, cementum attachment protein, erythropoietin, thrombopoietin, tumor necrosis factor (TNF), an interferon, a colony stimulating factor (CSF), or an interleukin, among others. After treatment with bioactive factors, the graft may be implanted into a patient.

Clinical usage of plasticized tissue and organ grafts includes direct implantation of the grafts without further processing following removal from the packaging, implantation following a brief washing in sterile isotonic saline or with water to remove any remaining traces of plasticizer associated with the immediate surfaces of the grafts, or by implantation following an extended (approximately one hour) washing with sterile isotonic saline or with water to remove as much plasticizer as possible. In the operating room, immediately prior to implantation, the plasticized tissue and organ grafts may also be further treated with modifying compositions to enhance their performance as a graft. Under any of the above described further processing of grafts, the materials properties of the plasticized grafts resemble those materials properties of fully or partially hydrated natural tissue (i.e. normal bone or soft tissue) or natural organ. The produced plasticized graft does not need to be rehydrated prior to clinical implantation, and the graft retains the strength and compressive/tensile properties of natural tissue. Plasticized tissue or organ grafts in which the plasticizer may be used to stabilize the matrix and load-bearing components of the tissue or organ graft, may also be directly implanted in a patient without rehydration/reconstitution.

The present invention also provides seeding cells onto the graft to enhance the performance of the graft, for example to help fusion of the graft to the surrounding tissues or organs. Before transplantation of the plasticized tissue or organ grafts into a patient, certain cells may be applied to the grafts. The cells may be derived from autologous or allogeneic sources. The cells may be differentiated cells include chondrocytes, osteoblasts, osteoclasts, endothelial cells, epithelial cells, fibroblasts. Additionally, the cells may be totipotent, pluripotent, multipotent, or progenitor stem cells. The stem cells may be derived from embryos, placenta, bone marrow, adipose tissue, blood vessel, amniotic fluid, synovial fluid, synovial membrane, peripheral blood, umbilical blood, menstrual blood, baby teeth, nucleus pulposus, brain, skin, hair follicle, intestinal crypt, neural tissue, skeletal muscle. The stem cells may be derived from genetic reprogramming of mature cells. The cells may be an isolated population or a mixture of differentiated cells and/or stem cells. The cells can be applied to the grafts with or without culture expansion.

To apply cells onto the plasticized graft for implantation, the graft may be briefly rinsed with and incubated in a medium to remove any remaining plasticizer and to condition the surface to facilitate cell attachment. The medium may be a fresh or a conditioned culture medium. After rinsing and incubating with the medium, cells may be applied to the graft.

Suitable surgical methods for implanting bone and soft tissue grafts and organ grafts into a patient are well known to those of ordinary skill in the art to which the invention pertains, and such methods are equally applicable to implantation of the present plasticized grafts. Those of ordinary skill in the art to which the present invention pertains can readily determine, select and employ suitable surgical methods without undue experimentation.

Further details of the process of the invention are presented in the examples that follow.

### EXAMPLES

### Example 1

### Processing of a Frozen Distal Femur

A. Cleaning and Processing: A frozen distal femur is selected and all of the soft tissue and periosteum is removed. The aft is then transected to the desired length using a STRYKER saw or a band saw. Each bisected piece is not more than 30 cm in length and is straight and contains no bone fragments. The surface cartilage is then removed from the femoral condyle with er a scalpel blade, periosteal elevator, or osteotome. The processing instructions dictate leaving the cartilage “on” when appropriate. Using a ⅜″ drill bit, the cut end of the shaft is drilled approximately 5 cm. The interior of the intramedullary canal is then thoroughly washed with a lavage system.

An intercalary fitting is then inserted by screwing the threaded, tapered end into the cut end of the graft. The vacuum tubing is assembled by securing one end of the tubing to the nipple end of the intercalary fitting. The other end of the tubing is secured to the piston driven pump. Finally, another section of vacuum tubing is secured to the other side of the piston pump. Approximately 4000 cc of a 1:100 dilution of ALLOWASH Solution is poured into the sterile flushing vessel. The ALLOWASH Solution is prepared by adding 4 cc of cleaning reagent to 3996 cc of sterile water. The flushing vessel is labeled ALLOWASH Solution. The open end of the second piece of vacuum tubing is placed into a graduated flask. The piston pump is set to “reverse” and the flow rate controller is set to 50%. The pump is turned on and at least 500 cc of the first solvent (ALLOWASH Solution) is drawn to waste. Thereafter, the open end of the second piece of vacuum tubing is removed from the graduated flask and placed into the sterile flushing vessel. The drive is maintained in the “reverse” position at 50%. The ALLOWASH Solution recirculates for a minimum of 15 minutes.

The 1:100 dilution of ALLOWASH solution is then decanted and approximately 4 liters of 3% hydrogen peroxide is added to the flushing vessel. The piston pump is set to reverse and the flow rate controller is set to 50%. The pump is then turned on and at least 500 cc of the 3% hydrogen peroxide solution is drawn to waste. Thereafter, the open end of the second piece of vacuum tubing is removed from the graduated flask and placed into the sterile flushing vessel. The drive is maintained in the reverse position at 50%. The hydrogen peroxide is then allowed to recirculate for a minimum of 15 minutes.

The hydrogen peroxide is then decanted and approximately 3980 cc of sterile water is added along with the entire contents of reconstituted vials of Bacitracin and Polymyxin B to the flushing vessel. The flushing vessel is clearly labeled “antibiotic.” The piston pump is then set to reverse and the flow rate controller is set at 50%. The pump is turned on and at least 500 cc of antibiotic solution is drawn to waste. The open end of the second piece of vacuum tubing is removed from the graduated flask and placed into the sterile flushing vessel. The drive is maintained in the reverse position at 50%. The antibiotic solution is allowed to recirculate for a minimum of 15 minutes.

B. Plasticization: The antibiotic solution is then decanted and approximately 4 liters of 70% isopropyl alcohol/30% glycerin USP is added to the flushing vessel. The flushing vessel is clearly labeled as 70% IPA/30% glycerin USP. The piston pump is set to reverse and the flow rate controller is set to 50%. The pump is turned on and at least 500 cc of IPA/glycerin USP solution is drawn to waste.

The open end of the second piece of vacuum tubing is removed from the graduated flask and placed into the sterile flushing vessel. The drive is maintained in the reverse position and the flow controller is set at 50%. The IPA/glycerin USP solution is allowed to recirculate for a minimum of 30 minutes and is then decanted. Thereafter, 4 liters of 30% glycerin USP in sterile water is added to the flushing vessel. The flushing vessel is labeled as glycerin USP washing solution. The piston pump is set to reverse and the flow rate controller is set to 50%. The pump is turned on and at least 500 cc of washing solution is drawn to waste.

The open end of the second piece of vacuum tubing is removed from the graduated flask and placed into the sterile flushing vessel. The drive is maintained in the reverse position and the flow rate controller is set to 50%. The washing solution is allowed to recirculate for a minimum of 15 minutes. Thereafter, the bone graft is removed from the flushing vessel and processed for freeze-drying as per standard operating procedure.

### Example 2

### Processing of a Frozen Distal Femur

A. Cleaning and Processing: A frozen distal femur is selected and all of the soft tissue and periosteum is removed using sharp dissection techniques and periosteal elevators. The graft is then transected to the desired length using a STRYKER saw or band saw. Each bisected piece is not more than 30 cm in length and is straight and contains no bone fragments. The surface cartilage is then removed from the femoral condyle with either a scalple blade, periosteal elevator, or osteotome. The processing instructions dictate leaving the cartilage “on” when appropriate. Using a ⅜″ drill bit, the cut end of the shaft is drilled approximately 5 cm. The interior of the intramedullary canal is then thoroughly washed with the lavage system.

An intercalary fitting is then inserted by screwing the threaded, tapered end into the cut end of the graft. The vacuum tubing is assembled by securing one end of the tubing to the nipple end of the intercalary fitting. The other end of the tubing is secured to the piston driven pump. Finally, another section of vacuum tubing is secured to the other side of the piston pump. Approximately 4000 cc of a 1:100 dilution of ALLOWASH Solution is poured into the sterile flushing vessel. The ALLOWASH Solution is prepared by adding 4 cc of cleaning reagent to 3996 cc of sterile water. The flushing vessel is labeled ALLOWASH Solution. The open end of the second piece of vacuum tubing is placed into a graduated flask. The piston pump is set to “reverse” and the flow rate controller is set to 50%. The pump is turned on and at least 500 cc of the first solvent (ALLOWASH Solution) is drawn to waste. Thereafter, the open end of the second piece of vacuum tubing is removed from the graduated flask and placed into the sterile flushing vessel. The drive is maintained in the “reverse” position at 50%. The ALLOWASH Solution recirculates for a minimum of 15 minutes.

B. Plasticization: The 1:100 dilution of ALLOWASH Solution is then decanted and approximately 4 liters of 3% hydrogen peroxide/30% glycerin USP is added to the flushing vessel. The piston pump is set to reverse and the flow rate controller is set to 50%. The pump is then turned on and at least 500 cc of the 3% hydrogen peroxide/glycerin USP solution is drawn to waste. Thereafter, the open end of the second piece of vacuum tubing is removed from the graduated flask and placed into the sterile flushing vessel. The drive is maintained in the reverse position. The hydrogen peroxide/glycerin USP is then allowed to recirculate for a minimum of 15 minutes.

The hydrogen peroxide/glycerin USP is then decanted and approximately 3980 cc of sterile water is added along with the entire contents of reconstituted vials of Bacitracin and Polymyxin B prepared in a water solution of 30% glycerin USP, to the flushing vessel. The flushing vessel is clearly labeled “antibiotic.” The piston pump is then set to reverse and the flow rate controller is set at 50%. The pump is turned on and at least 500 cc of antibiotic solution is drawn to waste. The open end of the second piece of vacuum tubing is removed from the graduated flask and placed into the sterile flushing vessel. The drive is maintained in the reverse position at 50%. The antibiotic solution is allowed to recirculate for a minimum of 15 minutes.

The antibiotic solution is then decanted and approximately 4 liters of 70% isopropyl alcohol/30% glycerin USP is added to the flushing vessel. The flushing vessel is clearly labeled as 70% IPA/30% glycerin USP. The piston pump is set to reverse and the flow rate controller is set to 50%. The pump is turned on and at least 500 cc of IPA/glycerin USP solution is drawn to waste.

The open end of the second piece of vacuum tubing is removed from the graduated flask and placed into the sterile flushing vessel. The drive is maintained in the reverse position and the flow controller is set at 50%. The PA/glycerin USP solution is allowed to recirculate for a minimum of 30 minutes and is then decanted. Thereafter, 4 liters of 30% glycerin USP in sterile water is added to the flushing vessel. The flushing vessel is labeled as glycerin USP washing solution. The piston pump is set to reverse and the flow rate controller is set to 50%. The pump is turned on and at least 500 cc of washing solution is drawn to waste.

The open end of the second piece of vacuum tubing is removed from the graduated flask and placed into the sterile flushing vessel. The drive is maintained in the reverse position and the flow rate controller is set to 50%. The washing solution is allowed to recirculate for a minimum of 15 minutes. Thereafter, the bone graft is removed from the flushing vessel and processed for freeze-drying as per standard operating procedure.

### Example 3

### Processing of a Frozen Distal Femur

A. Cleaning and Processing: A frozen distal femur is selected and all of the soft tissue and periosteum is removed using sharp dissection techniques and periosteal elevators. The graft is then transected to the desired length using a STRYKER saw or band saw. Each bisected piece is not more than 30 cm in length and is straight and contains no bone fragments. The surface cartilage is then removed from the femoral condyle with either a scalpel blade, periosteal elevator, or osteotome. The processing instructions dictate leaving the cartilage “on” when appropriate Using a ⅜″ drill bit, the cut end of the shaft is drilled approximately 5 cm. The interior of the intramedullary canal is then thoroughly washed with the lavage system.

An intercalary fitting is then inserted by screwing the threaded, tapered end into the cut end of the graft. The vacuum tubing is assembled by securing one end of the tubing to the nipple end of the intercalary fitting. The other end of the tubing is secured to the piston driven pump. Finally, another section of vacuum tubing is secured to the other side of the piston pump. Approximately 4000 cc of a 1:100 dilution of the ALLOWASH Solution is poured into the sterile flushing vessel. The ALLOWASH Solution is prepared by adding 4 cc of cleaning reagent. The flushing vessel is labeled as ALLOWASH Solution. The open end of the second piece of vacuum tubing is placed into a graduated flask. The piston pump is set to “reverse” and the flow rate controller is set to 50%. The pump is turned on and at least 500 cc of the first solvent (ALLOWASH Solution) is drawn to waste. Thereafter, the open end of the second piece of vacuum tubing is removed from the graduated flask and placed into the sterile flushing vessel. The drive is maintained in the “reverse” position at 50%. The ALLOWASH Solution recirculates for a minimum of 15 minutes.

The 1:100 dilution of the ALLOWASH Solution is then decanted and approximately 4 liters of 3% hydrogen peroxide is added to the flushing vessel. The piston pump is set to reverse and the flow rate controller is set to 50%. The pump is then turned on and at least 500 cc of the 3% hydrogen peroxide solution is drawn to waste. Thereafter, the open end of the second piece of vacuum tubing is removed from the graduated flask and placed it into the sterile flushing vessel. The drive is maintained in the reverse position at 50%. The hydrogen peroxide is then allowed to recirculate for a minimum of 15 minutes.

The hydrogen peroxide is then decanted and approximately 3980 cc of sterile water is added along with the entire contents of reconstituted vials of Bacitracin and Polymyxin B to the flushing vessel. The flushing vessel is clearly labeled “antibiotic.” The piston pump is then set to reverse and the flow rate controller is set at 50%. The pump is turned on and at least 500 cc of antibiotic solution is drawn to waste. The open end of the second piece of vacuum tubing is removed from the graduated flask and placed into the sterile flushing vessel. The drive is maintained in the reverse position at 50%. The antibiotic solution is allowed to recirculate for a minimum of 15 minutes.

The antibiotic solution is then decanted and approximately 4 liters of 70% isopropyl alcohol (IPA) is added to the flushing vessel. The flushing vessel is labeled as 70% IPA. The piston pump is set to reverse and the flow rate controller is set to 50%. The pump is turned on and at least 500 cc of IPA solution is drawn to waste. The open end of the second piece of vacuum tubing is removed from the graduated flask and placed into the sterile flushing vessel. The drive is maintained in the reverse position and the flow controller is set to 50%. The IPA recirculates for a minimum of 15 minutes. The IPA solution is then decanted and 4 liters of sterile water is added to the flushing vessel. The flushing vessel is labeled as “washing solution.” The piston pump is set to reverse and the flow rate controller is set to 50%. The pump is turned on and at least 500 cc of washing solution is drawn to waste.

The open end of the second piece of vacuum tubing is removed from the graduated flask and placed into the sterile flushing vessel. The drive is maintained in the reverse position and the flow rate controller is set to 50%. The washing solution recirculates for a minimum of 15 minutes. The bone graft is removed from the flushing vessel and processed for freeze-drying as per standard operating procedure.

B. Plasticization: The freeze-dried bone graft(s) are then placed into sterile glycerin USP such that they are totally immersed in the viscous glycerol. Vacuum (10 to 500 mTorr, preferably 100 to 200 mTorr) is applied to the container until bubbles cease to exit the bone graft (about 5 to 60 minutes depending on the size and configuration of the bone graft, preferably about 20 to 30 minutes). The bone graft(s) are then removed from the glycerin USP solution and placed into an appropriate centrifuge container on top of a graft support.

The bone graft(s) are centrifuged at about 1000 to 2000 rpm until the glycerol ceases to exit the bone graft and accumulate in the bottom of the centrifuge container (usually 5 to 60 minutes depending on the size and configuration of the bone graft, preferably about 5 to 15 minutes). The bone graft(s) are then removed from their respective centrifuge containers and packaged for distribution.

### Example 4

### Processing Cloward Dowels

A. Cleaning and Processing: Graft material is selected and all of the soft tissue and periosteum is removed from the distal femur, proximal and distal tibia, and cartilage is removed from the site. The femur is transected 10-15 cm above the femoral condyles and the distal femoral condyles are bisected. Transect the proximal tibia 10-15 cm below the tibial plateau. The distal femur or proximal tibia is placed in a PAN-A-VISE. This is accomplished by removing a section of the diaphysis, allowing the vise jaws to grip the tissue securely. The Cloward set (12, 14, 16, 18, or 20 mm) is then assembled: 1. Place the extractor assembly within the cutter shaft; 2. Screw the cutter assembly onto the shaft with the aid of the Cloward set wrench; 3. Screw the set-point onto the extractor assembly; and 4. Insert the shaft of the Cloward set into the ⅜″ variable speed drill and tighten the chuck with the key. The set-point is then placed and locked at the forward aspect of the cutter.

The apparatus is then placed on the tissue to be fashioned. Drilling is commenced at a moderate speed. After the set-point has made a deep cut in the tissue, and the teeth have begun to cut into the tissue, drilling is stopped, and the set-point apparatus is unlocked. Drilling is continued using the marks created as a guide.

The Cloward(s) are then removed from the tissue block. A STRYKER saw or band saw is then used to remove the cut grafts after all have been cut. Any cartilage is then trimmed from the cortical face of the Cloward(s) using a scalpel and a #10 blade. The distal end of the graft is then trimmed perpendicular to the body of the graft with a band saw making sure the fashioned graft is at least 15 mm long. The Cloward(s) are cleansed using pulsatile water apparatus. If the surface marrow is not easily removed, dry spin the graft(s) at 2600 rpm for 3 minutes.

The Cloward(s) are then placed in a sterile container with hydrogen peroxide (3%) at 37 to 44° C. The container is sealed and the container is placed into the centrifuge. The centrifuge is then balanced. The grafts are then centrifuged at 2600 rpm for 15 minutes. The tissue is removed from the centrifuge and the rafts are placed into an ultrasonic cleaner. Equal volumes of ALLOWASH Solution, hydrogen peroxide (3%), and antibiotics are added to the ultrasonic cleaner and sonicate the tissue at 37-44° C. for a minimum of 1 hour. Thereafter, the tissue is removed from the ultrasonic cleaner.

The mixture is decanted and a sterile glass container is filled with fresh 3% hydrogen peroxide. The grafts are then placed in the container, the top is sealed and the container is taken to the large ultrasonic cleaner. The grafts are then sonicated for 90 minutes. Thereafter, the grafts are incubated overnight at 37-44° C. (minimum of 6 hours, preferably 12 to 18 hours).

B. Plasticization: After incubation, the hydrogen peroxide is decanted and the basin is filled with 70% isopropyl alcohol/30% glycerin USP and the grafts are incubated at room temperature for a minimum of 30 minutes. Thereafter, the isopropyl alcohol/glycerin USP solution is decanted and the container is filled with warm 30% glycerin USP in water. The grafts are incubated for a minimum of 30 minutes. Methods of incubation include for example: soaking.

The glycerin solution is then decanted and the Cloward dowels are removed from the container. The Cloward dowels are then placed into a sterile container. The container is sealed and placed into the centrifuge. The centrifuge is balanced and the grafts are centrifuged for 3-5 minutes to dry, and the remaining solution is removed.

The width and length of the Cloward(s) are measured, raft identification numbers are assigned, and the information is recorded on the “Tissue Processing Log Worksheet”. One graft is then placed into a glass, 120 cc bottle and the printed label is affixed with the unique numeric designator. This step is repeated until all deposits are bottled. The bottled grafts are either frozen and packaged, or frozen and freeze-dried and packaged.

### Example 5

### Processing Cloward Dowels

A. Cleaning and Processing: Graft material is selected and all of the soft tissue and periosteum is removed from the distal femur, proximal and distal tibia, and cartilage is removed from the site. The femur is transected 10-15 cm above the femoral condyles and the distal femoral condyles are bisected. Transect the proximal tibia 10-15 cm below the tibial plateau. The distal femur or proximal tibia is placed in a PAN-A-VISE. This is accomplished by removing a section of the diaphysis, allowing the vise jaws to grip the tissue securely. The Cloward set (12, 14, 16, 18, or 20 mm) is then assembled: 1. Place the extractor assembly within the cutter shaft; 2. Screw the cutter assembly onto the shaft with the aid of the Cloward set wrench; 3. Screw the set-point onto the extractor assembly; and 4. Insert the shaft of the Cloward set into the ⅜″ variable speed drill and tighten the chuck with the key. The set-point is then placed and locked at the forward aspect of the cutter.

The apparatus is then placed on the tissue to be fashioned. Drilling is commenced at a moderate speed. After the set-point has made a deep cut in the tissue, and the teeth have begun to cut into the tissue, drilling is stopped, and the set-point apparatus is unlocked. Drilling is continued using the marks created as a guide.

The Cloward(s) are then removed from the tissue block. A STRYKER saw or band saw is then used to remove the cut grafts after all have been cut. Any cartilage is then trimmed from the cortical face of the Cloward(s) using a scalpel and a #10 blade. The distal end of the graft is then trimmed perpendicular to the body of the graft with a band saw making sure the fashioned graft is at least 15 mm long. The Cloward(s) are cleansed using pulsatile water apparatus. If the surface marrow is not easily removed, dry spin the graft(s) at 2600 rpm for 3 minutes.

B. Plasticization: The Cloward(s) are then placed in a sterile container with hydrogen peroxide (3%) and glycerin USP (30%) at 37 to 44° C. The container is sealed and the container is placed into the centrifuge. The centrifuge is then balanced. The grafts are then centrifuged at 2600 rpm for 15 minutes. The tissue is removed from the centrifuge and the grafts are placed into an ultrasonic cleaner. Equal volumes of ALLOWASH Solution, hydrogen peroxide (3%), 30% glycerin USP, and antibiotics are added to the ultrasonic cleaner and the tissue is sonicated at 37-44° C. for a minimum of 1 hour. Thereafter, the tissue is removed from the ultrasonic cleaner.

The mixture is decanted and a sterile glass container is filled with fresh 3% hydrogen peroxide/30% glycerin USP. The grafts are then placed in the container, the top is sealed and the container is taken to the large ultrasonic cleaner. The grafts are then sonicated for 90 minutes.

Thereafter, the grafts are incubated overnight at 37-44° C. (minimum of 6 hours, preferably 12 to 18 hours).

After incubation, the hydrogen peroxide is decanted and the basin is filled with 70% isopropyl alcohol/30% glycerin USP and the grafts are incubated at room temperature for a minimum of 30 minutes. Thereafter, the isopropyl alcohol/glycerin USP solution is decanted and the container is filled with warm 30% glycerin USP in water. The grafts are incubated for a minimum of 30 minutes. Methods of incubation include for example: soaking and mild agitation.

The glycerin solution is then decanted and the Cloward dowels are removed from the container. The Cloward dowels are then placed into a sterile container. The container is sealed and placed into the centrifuge. The centrifuge is balanced and the grafts are centrifuged for 3-5 minutes to dry, and the remaining solution is removed.

The width and length of the Cloward(s) are measured, graft identification numbers are assigned, and the information is recorded on the “Tissue Processing Log Worksheet”. One graft is then placed into a glass, 120 cc bottle and the printed label is affixed with the unique numeric designator. This step is repeated until all deposits are bottled. The bottled grafts are either frozen and packaged, or frozen and freeze-dried and packaged.

### Example 6

### Processing Cloward Dowels

A. Cleaning and Processing: Graft material is selected and all of the soft tissue and periosteum is removed from the distal femur, proximal and distal tibia, and cartilage is removed from the site. The femur is transected 10-15 cm above the femoral candles and the distal femoral candles are bisected. The proximal tibia is transected 10-15 cm below the tibial plateau. The distal femur or proxmal tibia is placed in a PAN-A-VISE. This is accomplished by removing a section of the diaphysis, allowing the vise jaws to grip the tissue securely. The Cloward set (12, 14, 16, 18, or 20 mm) is then assembled: 1. place the extractor assembly within the cutter shaft; 2. screw the cutter assembly onto the shaft with the aid of the Cloward set wrench; 3. screw the set-point onto the extractor assembly, and 4. insert the shaft of the Cloward set into the ⅜″ variable speed drill and tighten the chuck with the key. The set-point is then placed and locked at the forward aspect of the cutter.

The apparatus is then placed on the tissue to be fashioned. Drilling is commenced at a moderate speed. After the set-point has made a deep cut in the tissue, and the teeth have begun to cut into the tissue, drilling is stopped, and the set-point apparatus is unlocked. Drilling is continued using the marks created as a guide.

The Clowards are then removed from the tissue block. A STRYKER saw or a band saw is then used to remove the cut grafts after all have been cut. Any cartilage is then trimmed from the cortical face of the Clowards using a scalpel and a #10 blade. The distal end of the graft is then trimmed perpendicular to the body of the graft with a saw making sure the fashioned graft is at least 15 mm long. The Clowards are cleansed using pulsatile water lavage. If the surface marrow is not easily removed, dry spin the grafts at 2600 rpm for 3 minutes. The grafts may optionally be further processed according to methods for example, as described above.

The Clowards are then placed in a sterile container with hydrogen peroxide (3%) at 37-44° C. The container is sealed and the container is placed into the centrifuge. The centrifuge is then balanced. The grafts are centrifuged at 2600 rmp for 15 minutes. The tissue is removed from the centrifuge and the grafts are placed into an ultrasonic cleaner. Equal volumes of ALLOWASH Solution, hydrogen peroxide (3%), and antibiotics are added to the ultrasonic cleaner and the tissue is sonicated at 37-44° C. for a minimum of 1 hour. Thereafter, the tissue is removed from the ultrasonic cleaner.

The mixture is decanted and a sterile glass container is filled with fresh 3% hydrogen peroxide. The grafts are then placed in the container, the top is sealed and the container is taken to the large ultrasonic cleaner. The grafts are then sonicated for 90 minutes. Thereafter, the grafts are incubated overnight at 37-44° C. (minimum of 6 hours, preferably 12 to 18 hours).

After incubation, the hydrogen peroxide is decanted and the basin is filled with 70% isopropyl alcohol and the grafts are incubated at room temperature for a minimum of 30 minutes. Thereafter, the isopropyl alcohol and the grafts are incubated at room temperature for a minimum of 30 minutes. Thereafter, the isopropyl alcohol is decanted and the container is filled with warm sterile water. The grafts are incubated for a minimum of 30 minutes. Methods of incubation include for example, soaking, mild agitation, sonication, and centrifugation.

The wash solution is then decanted and the Cloward dowels are removed from the container. The Cloward dowels are then placed into a sterile container. The container is sealed and placed into the centrifuge. The centrifuge is balanced. The grafts are then centrifuged for 3-5 minutes to dry and the remaining solution is removed.

The width and length of the Cloward dowels are measured, graft identification numbers are assigned, and the information is recorded on the “Tissue Processing Log Worksheet.” One graft is then placed into a glass, 120 cc bottle and the printed label is affixed with the unique numeric designator. This step is repeated until all deposits are bottled. The bottled grafts are either frozen and packaged, or frozen and freeze-dried and packaged.

B. Plasticization: Viscous glycerol is then added to each bottle sufficient to cover the graft and vacuum (10 to 500 mTorr) is applied to each bottle until the air ceases to exit the grafts (usually 5-20 minutes depending on graft type, however, complete air removal may take up to 24 hours). The grafts are then removed from the bottles and placed into a centrifuge container. The grafts are centrifuged for 10 sec-5 min to remove excess glycerol. The bottled grafts are either packaged or placed under vacuum and packaged.

### Example 7

### Processing Cloward Dowels

A. Cleaning and Processing: Graft material is selected and all of the soft tissue and periosteum is removed from the distal femur, proximal and distal tibia, and cartilage is removed from the site. The femur is transected 10-15 cm above the femoral condyles and the distal femoral condyles are bisected. Transect the proximal tibia 10-15 cm below the tibial plateau. The distal femur or proximal tibia is placed in a Pan-A-Vise™. This is accomplished by removing a section of the diaphysis, allowing the vise jaws to grip the tissue securely. The Cloward set (12, 14, 16, 18, or 20 mm) is then assembled: 1. Place the extractor assembly within the cutter shaft; 2. Screw the cutter assembly onto the shaft with the aid of the Cloward set wrench; 3. Screw the set-point onto the extractor assembly; and 4. Insert the shaft of the Cloward set into the ⅜″ variable speed drill and tighten the chuck with the key. The set-point is then placed and locked at the forward aspect of the cutter.

The apparatus is then placed on the tissue to be fashioned. Drilling is commenced at a moderate speed. After the set-point has made a deep cut in the tissue, and the teeth have begun to cut into the tissue, drilling is stopped, and the set-point apparatus is unlocked. Drilling is continued using the marks created as a guide.

The Cloward(s) are then removed from the tissue block. A STRYKER saw or band saw is then used to remove the cut grafts after all have been cut. Any cartilage is then trimmed from the cortical face of the Cloward(s) using a scalpel and a #10 blade. The distal end of the graft is then trimmed perpendicular to the body of the graft with a band saw making sure the fashioned graft is at least 15 mm long. The Cloward(s) are cleansed using pulsatile water apparatus. If the surface marrow is not easily removed, dry spin the graft(s) at 2600 rpm for 3 minutes.

B. Plasticization. The Cloward(s) are then placed in a sterile container with hydrogen peroxide (3%)/glycerin USP 30% at 37 to 44° C. The container is sealed and the container is placed into the centrifuge. The centrifuge is then balanced. The grafts are then centrifuged at 2600 rpm for 15 minutes. The tissue is removed from the centrifuge and the grafts are placed into an ultrasonic cleaner. Equal volumes of ALLOWASH Solution, hydrogen peroxide (3%), and antibiotics are added to the ultrasonic cleaner and sonicate the tissue at 37-44° C. for a minimum of 1 hour. Thereafter, the tissue is removed from the ultrasonic cleaner.

The mixture is decanted and a sterile glass container is filled with fresh 3% hydrogen peroxide. The grafts are then placed in the container, the top is sealed and the container is taken to the large ultrasonic cleaner. The grafts are then sonicated for 90 minutes. Thereafter, the grafts are incubated overnight at 37-44° C. (minimum of 6 hours, preferably 12 to 18 hours).

After incubation, the hydrogen peroxide is decanted and the basin is filled with 70% isopropyl alcohol/30% glycerin USP and the grafts are incubated at room temperature for a minimum of 30 minutes. Thereafter, the isopropyl alcohol/glycerin USP solution is decanted and the container is filled with warm 30% glycerin USP in sterile water. The grafts are incubated for a minimum of 30 minutes. Methods of incubation include for example: soaking and mild agitation.

The solution is then decanted and the Cloward dowels are removed from the container. The Cloward dowels are then placed into a sterile container. The container is sealed and placed into the centrifuge. The centrifuge is balanced. The grafts are then centrifuged for 3-5 minutes to dry and the remaining solution is removed.

The width and length of the Cloward(s) are measured, graft identification numbers are assigned, and the information is recorded on the “Tissue Processing Log Worksheet”. One graft is then placed into a glass, 120 cc bottle and the printed label is affixed with the unique numeric designator. This step is repeated until all deposits are bottled. The bottled grafts are either frozen and packaged, or frozen and freeze-dried and packaged.

### Example 8

### Processing of an Iliac Crest Wedge

A. Cleaning and Processing: The soft tissue, periosteum, and cartilage is removed from an ilium. The ilium is placed in a PAN-A-VISE by removing a section of the ilium, allowing the vise jaws to grip the tissue securely. A STRYKER saw is assembled with parallel cutting blades (12, 14, 16, 18, or 20 mm). The set-point at the forward aspect of the cutter is placed and locked. The apparatus is placed on the tissue to be fashioned and cutting is begun at a moderate speed.

After the set-point has made a deep cut in the tissue, and the teeth have begun to cut into the tissue, cutting is stopped, and the set-point apparatus is checked. Cutting is continued using the marks created as a guide. A STRYKER saw or band saw is then used to remove the cut grafts after all have been cut.

Any cartilage is trimmed from the cortical face of the grafts(s) using a scalpel and a #10 blade. The distal end of the graft perpendicular to the body of the graft is trimmed with a band saw making sure the fashioned graft is at least 15 mm long. The Grafts is then cleansed using a pulsatile water apparatus. If the surface marrow is not easily removed, the graft(s) is dry spun at 2600 rpm for 3 minutes.

B. Plasticization: The Iliac Crest Wedge(s) are then placed in a sterile container with hydrogen peroxide (3%) and glycerin USP (30%) at 37 to 44° C. The container is sealed and placed into the centrifuge. The centrifuge is balanced. The grafts are then centrifuged at 2600 rpm for 15 minutes. The tissue is removed from the centrifuge and the grafts are placed into the ultrasonic cleaner. Equal volumes of ALLOWASH Solution, hydrogen peroxide (3%), glycerin USP (30%), and antibiotics are added to the ultrasonic cleaner, and the grafts are sonicated at 37-44° C. for a minimum of 1 hour.

The tissue is then removed from the ultrasonic cleaner. The mixture is decanted and a sterile glass container is filled with fresh 3% hydrogen peroxide/30% glycerin USP. The grafts are placed in the container, the top is sealed and the container is taken to a large ultrasonic cleaner. The grafts are sonicated for 90 minutes. Thereafter, the grafts are incubated overnight at 37-44° C. (minimum of 6 hours, preferably for 12 to 18 hours). Methods of incubation include for example: soaking and mild agitation.

The hydrogen peroxide/glycerin USP is then decanted and the basin is filled with 70% isopropyl alcohol/30% glycerin USP. The grafts are then incubated at room temperature for a minimum of 30 minutes. The isopropyl alcohol/glycerin USP solution is then decanted and the container is filled with warm 30% glycerin USP in water. The grafts are incubated for a minimum of 30 minutes.

The glycerin USP solution is then decanted and the Iliac Crest Wedges are removed from the container. The Iliac Crest Wedges are then placed into a sterile container. The container is sealed and placed into the centrifuge. The centrifuge is balanced and the grafts are centrifuged for 3-5 minutes to dry and the remaining solution is removed.

The width and length of the Wedges are measured, graft identification numbers are assigned, and the information is recorded on the “Tissue Processing Log Worksheet”. One graft is then placed into a glass, 120 cc bottle and the printed label is affixed with the unique numeric designator. This step is repeated until all deposits are bottled. The bottled grafts are either frozen and packaged, or frozen and freeze-dried and packaged.

### Example 9

### Processing of Fascia Lata

A. Cleaning and Processing: Any remaining muscle tissue is removed from the fascia lata. The fascia is placed with the subcutaneous layer uppermost, on a clean, drape towel. Using blunt dissection techniques, all of the fat and extraneous soft tissue is removed from the graft material. The graft is kept moist with sterile water to prevent desiccation during processing.

Any torn fibers are removed from the edges of the graft material, and a graft rectangular in shape is created. The graft(s) are then placed in a basin containing a 1:100 dilution of ALLOWASH Solution or other surfactant(s) for at least 15 minutes. The basin is labeled as ALLOWASH Solution. The time of exposure is recorded on the Tissue Processing Log Worksheet.

B. Plasticization: The graft(s) are placed into an empty basin labeled “Rinse”. The graft(s) are rinsed three times with copious amounts of sterile water to remove any residual detergents. Any sterile water which accumulates in the Rinse basin is discarded. The number of rinses is recorded on the Tissue Processing Log Worksheet. The fashioned graft(s) are then placed in the basin containing U.S.P. grade 70% isopropyl alcohol containing 30% glycerin USP for 2-5 minutes. The basin is labeled IPA/Glycerin. The time of exposure to the alcohol/glycerin USP solution is recorded in the Tissue Processing Log Worksheet.

The graft(s) are then placed into the basin containing the antibiotic solution in 30% glycerin USP for at least 15 minutes. The basin is labeled as Antibiotics/Glycerin USP. The exposure time to the antibiotics/glycerin USP is recorded on the Tissue Processing Log Worksheet. The graft(s) are then thoroughly soaked by immersing each deposit into sterile 30% glycerin USP in deionized/distilled water for a minimum of 5 minutes to remove excess antibiotics. Enough sterile glycerin USP solution is needed to cover the graft(s). The basin is labeled as Rinse. The time of exposure to the glycerin USP rinse solution is recorded on the Tissue Processing Log Worksheet.

The fashioned graft(s) are then placed on sterile fine mesh gauze, and the gauze is trimmed to just beyond the edges of the graft. The width and length of the graft(s) is measured to the nearest tenth of a centimeter. The graft(s) are assigned identification numbers and this information is recorded on the Tissue Processing Log Worksheet. The graft and gauze is then rolled into a tube and graft material is then placed into glass, 120 ml bottles, and the printed label is affixed with the unique numeric designator. This step is repeated until all deposits are bottled. The graft material is now ready for wrapping and freeze-drying or dehydrating.

### Example 10

### Processing Pericardium

A. Cleaning and Processing: The pericardial tissue is rinsed of any blood or pericardial fluid in sterile water in the basin labeled Rinse. The pericardium is then placed on a clean drape towel. Using blunt dissection techniques, all of the fat and extraneous soft tissue is removed from the graft material. The graft is kept moist with sterile water to prevent desiccation during processing. Any torn fibers are removed from the edges of the graft material, and a graft rectangular in shape is created. The graft(s) are then placed in a basin containing a 1:100 dilution of ALLOWASH Solution or other surfactant(s) for at least 15 minutes. The basin is labeled as ALLOWASH Solution. The time of exposure is recorded on the Tissue Processing Log Worksheet.

The graft(s) are placed into an empty basin labeled “Rinse”. The graft(s) are rinsed three times with copious amounts of sterile water to remove any residual detergents. Any sterile water which accumulates in the Rinse basin is discarded. The number of rinses is recorded on the Tissue Processing Log Worksheet. The fashioned graft(s) are then placed in the basin containing U.S.P. grade 70% isopropyl alcohol containing 30% glycerin USP for 2-5 minutes. The basin is labeled IPA/Glycerin. The time of exposure to the alcohol/glycerin USP solution is recorded in the Tissue Processing Log Worksheet.

B. Plasticization: The graft(s) are then placed into the basin containing the antibiotic solution in 30% glycerin USP for at least 15 minutes. The basin is labeled as Antibiotics/Glycerin USP. The exposure time to the antibiotics/glycerin USP is recorded on the Tissue Processing Log Worksheet. The graft(s) are then thoroughly soaked by immersing each deposit into sterile 30% glycerin USP in deionized/distilled water for a minimum of 5 minutes, preferably from 10 to 15 minutes, to remove excess antibiotics. Enough sterile glycerin USP-solution is needed to cover the graft(s). The basin is labeled as Rinse. The time of exposure to the glycerin USP rinse solution is recorded on the Tissue Processing Log Worksheet.

The fashioned graft(s) are then placed on sterile fine mesh gauze, and the gauze is trimmed to just beyond the edges of the graft. The width and length of the graft(s) is measured to the nearest tenth of a centimeter. The graft(s) are assigned identification numbers and this information is recorded on the Tissue Processing Log Worksheet. The graft and gauze is then rolled into a tube and graft material is then placed into glass, 120 ml bottles, and the printed label is affixed with the unique numeric designator. This step is repeated until all deposits are bottled. The graft material is now wrapped and placed in a freeze dryer or dehydrated.

### Example 11

### Processing of a VG2 Cervical Grafts

A. Cleaning and Processing: VG2 cervical composite cortical bone grafts were cut, assembled and cleaned.

B. Plasticization: The cleaned grafts were dried and placed into a container with 80% v/v glycerol at room temperature. The grafts were completely covered with glycerol/alcohol. The containers were then sealed ans placed in a centrifuge. The centrifuge was balanced and the grafts were then centrifuged at 1200 rcf for 45 min-5 hrs. The containers were removed from the centrifuge and excess glycerol was poured off. The grafts were removed to a new container and blotted dry or dry spun at 1200 rcf for 10 seconds. The grafts were then vacuum packed and stored at room temperature.

### Example 12

### Processing Pericardium

A. Cleaning and Processing: The pericardial tissue is rinsed of any blood or pericardial fluid in sterile water in a container. The pericardium is then placed on a clean drape towel. Using blunt dissection techniques, all of the fat and extraneous soft tissue is removed from the graft material. The graft is kept moist with sterile water to prevent desiccation during processing. Any torn fibers are removed from the edges of the graft material, and a graft rectangular in shape is created. The graft(s) are rinsed three times with copious amounts of sterile water. The number of rinses is recorded on the Tissue Processing Log Worksheet.

The pericardial tissue is then contacted with glutaraldehyde (0.5% by weight, pH approximately 7.4) for 1 day at 37° C. The crosslinked pericardial tissue is then transferred into a glutaraldehyde with a concentration of 0.25% by weight in a sealed container and sterilize for 3 days at 37° C. The graft(s) are rinsed with sterile water to remove any residual glutaraldehyde.

B. Plasticization. Any sterile water which accumulates in the rinse container is discarded. The fashioned graft(s) are then placed in a container containing sterile 75-80% glycerin USP for 12-36 hours. The treated graft(s) are then transferred into a sealed package. Optionally the packaged graft(s) is further sterilized by gamma irradiation.

All of the publications and patent applications cited herein are hereby incorporated by reference into the present disclosure. It will be appreciated by those skilled in the art that various modifications can be made without departing from the essential nature thereof.

1. A plasticized tissue or organ suitable for transplantation comprising one or more plasticizers.

2. The plasticized tissue or organ of claim 1, wherein the tissue or organ is selected from the group consisting of bone, bone graft, heart valve, venous valve, blood vessel, ureter, skin, dermis, small intestine, large intestine, periosteum, nerve, menisci, cartilage, non-valve patch, muscle, dura, pericardium, fascia, tendon, and ligament.

3. The plasticized tissue or organ of claim 1, wherein the tissue or organ is derived from a mammal.

4. The plasticized tissue or organ of claim 3, wherein the mammal is a human, a cow, a pig, a goat, a dog, a horse, or a sheep.

5. The plasticized tissue or organ of claim 1, wherein prior to plasticization, the tissue or organ is processed by cleaning, treatment, or a combination thereof.

6. The plasticized tissue or organ of claim 5, wherein cleaning the tissue further comprises disinfecting the tissue.

7. The plasticized tissue or organ of claim 1, wherein the plasticizer is a polyol or a fatty acid.

8. The plasticized tissue or organ of claim 7, wherein the plasticizer is glycerol.

9. A method of producing a plasticized tissue or organ comprising incubating a tissue or organ with a plasticizer composition containing one or more plasticizers.

10. The method of claim 9, wherein the tissue or organ is selected from the group consisting of bone, heart valve, venous valve, blood vessel, ureter, skin, dermis, small intestine, large intestine, periosteum, nerve, menisci, cartilage, non-valve patch, muscle, dura, pericardium, fascia, tendon, and ligament.

11. The method of claim 9, wherein the tissue or organ is derived from a mammal.

12. The method of claim 11, wherein the mammal is a human, a cow, a pig, a dog, a horse, a goat, or a sheep.

13. The method of claim 9, wherein the plasticizer is a polyol or a fatty acid.

14. The method of claim 13, wherein the plasticizer is glycerol.

15. The method of claim 9, wherein the plasticizer composition further comprises one or more biocompatible solvents.

16. The method of claim 15, wherein the biocompatible solvent is an alcohol.

17. The method of claim 16, wherein the plasticizer is glycerol.

18. The method of claim 9, wherein the plasticizer composition further comprises a permeation enhancer.

19. The method of claim 9, wherein the tissue or organ is skin or dermis and the plasticizer composition comprises between about 10% v/v to 20% v/v plasticizer.

20. The method of claim 19, wherein the plasticizer composition comprises about 15% v/v plasticizer.

21. The method of claim 9, wherein the tissue is vein, artery, or non-valve patch and the plasticizer composition comprises between about 75% v/v to 85% v/v plasticizer.

22. The method of claim 21, wherein the plasticizer composition comprises about 80% v/v plasticizer.

23. The method of claim 9, wherein the tissue is bone and the plasticizer is greater than 70% v/v plasticizer.

23. The method of claim 9, wherein the tissue is bone and the plasticizer composition comprises between about 75% v/v to 80% v/v plasticizer.

25. The method of claim 23, wherein the plasticizer composition comprises about 77% v/v plasticizer.

26. The method of claim 9, wherein the method further comprises cleaning and/or treating the tissue or organ prior to plasticization.

27. The method of claim 26, wherein cleaning further comprises disinfecting the tissue or organ.

28. The method of claim 26, wherein treating the tissue or organ comprises incubating the tissue or organ with a modifying composition to enhance its performance as a graft.

29. The method of claim 28, wherein the modifying composition comprises one or more bioactive factors, analgesics, antithrombics, cell adhesion molecules, chemotactants, or combinations thereof.

30. The method of claim 9, further comprising dehydrating the plasticized tissue or organ after plasticization.

31. The method of claim 9, further comprising sterilizing the plasticized tissue or organ after plasticization.

32. The method of claim 9, further comprising packing the plasticized tissue or organ for storage after plasticization.

33. A method of transplanting a tissue or organ into a subject comprising surgically implanting the plasticized tissue or organ of claim 1 into a subject in need thereof.

34. The method of claim 33, wherein the plasticized tissue or organ is derived from an allogenic source, autogenic source, or a xenogenic source.

35. The method of claim 33, further comprising prior to implantation, incubating the plasticized tissue or organ with a medium to condition the tissue for cell seeding; seeding the cells onto a graft; and implanting the graft in a subject in need thereof.

36. A method of preventing a tissue or an organ for implantation from fracturing comprising incubating a tissue or organ with a plasticizer composition comprising one or more plasticizers under conditions that allow the one or more plasticizers to impregnate the tissue.

37. A method to enable storage of a tissue or an organ at room or low temperature comprising incubating the tissue or organ with a plasticizer composition comprising one or more plasticizers under conditions that allow the one or more plasticizers to impregnate the tissue.

38. A method of stabilizing a tissue or an organ in a dehydrated state comprising incubating a dehydrated tissue or organ with a plasticizer composition comprising one or more plasticizers under conditions that allow the one or more plasticizers to impregnate the tissue.
